SEQUENCE LISTING (26 sequences)

SEQ ID NO: 1 - protein, 653 aa, Homo sapiens
  Human IDS
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 2 - protein, 24 aa, Artificial Sequence
  Oligodendrocyte-myelin glycoprotein (hOMG) signal peptide
MEYQILKMSLCLFILLFLTPGILC

SEQ ID NO: 3 - protein, 31 aa, Artificial Sequence
  Cellular repressor of E1A-stimulated genes 2 (hCREG2) signal peptide
MSVRRGRRPARPGTRLSWLLCCSALLSPAAG

SEQ ID NO: 4 - protein, 28 aa, Artificial Sequence
  V-set and transmembrane domain containing 2B (hVSTM2B) signal peptide
MEQRNRLGALGYLPPLLLHALLLFVADA

SEQ ID NO: 5 - protein, 29 aa, Artificial Sequence
  Protocadherin alpha-1 (hPCADHA1) signal peptide
MVFSRRGGLGARDLLLWLLLLAAWEVGSG

SEQ ID NO: 6 - protein, 19 aa, Artificial Sequence
  FAM19A1 (TAFA1) signal peptide
MAMVSAMSWVLYLWISACA

SEQ ID NO: 7 - protein, 26 aa, Artificial Sequence
  VEGF-A signal peptide
MNFLLSWVHWSLALLLYLHHAKWSQA

SEQ ID NO: 8 - protein, 29 aa, Artificial Sequence
  Fibulin-1 signal peptide
MERAAPSRRVPLPLLLLGGLALLAAGVDA

SEQ ID NO: 9 - protein, 19 aa, Artificial Sequence
  Vitronectin signal peptide
MAPLRPLLILALLAWVALA

SEQ ID NO: 10 - protein, 18 aa, Artificial Sequence
  Complement Factor H signal peptide
MRLLAKIICLMLWAICVA

SEQ ID NO: 11 - protein, 19 aa, Artificial Sequence
  Opticin signal peptide
MRLLAFLSLLALVLQETGT

SEQ ID NO: 12 - protein, 18 aa, Artificial Sequence
  Albumin signal peptide
MKWVTFISLLFLFSSAYS

SEQ ID NO: 13 - protein, 18 aa, Artificial Sequence
  Chymotrypsinogen signal peptide
MAFLWLLSCWALLGTTFG

SEQ ID NO: 14 - protein, 20 aa, Artificial Sequence
  Interleukin-2 signal peptide
MYRMQLLSCIALILALVTNS

SEQ ID NO: 15 - protein, 15 aa, Artificial Sequence
  Trypsinogen-2 signal peptide
MNLLLILTFVAAAVA

SEQ ID NO: 16 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - protein, 735 aa, Artificial Sequence [sequence omitted]

SEQ ID NO: 18 - protein, 736 aa, Artificial Sequence
  AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 19 - protein, 734 aa, Artificial Sequence
  AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 20 - protein, 724 aa, Artificial Sequence
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 736 aa, Artificial Sequence
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - protein, 737 aa, Artificial Sequence
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 23 - protein, 738 aa, Artificial Sequence
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - protein, 736 aa, Artificial Sequence
  hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 25 - protein, 736 aa, Artificial Sequence
  hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2019/0358303 A1
(43) Pub. Date: Nov. 28, 2019

(54) TREATMENT OF MUCOPOLYSACCHARIDOSIS I WITH FULLY-HUMAN GLYCOSYLATED HUMAN ALPHA-L-IDURONIDASE (IDUA)

(71) Applicant: REGENXBIO Inc., Rockville, MD (US)

(72) Inventors: Stephen Yoo, Bethesda, MD (US); Rickey Robert Reinhardt, Silver Spring, MD (US); Curran Matthew Simpson, Frederick, MD (US); Zhuchun Wu, North Potomac, MD (US)

(73) Assignee: REGENXBIO Inc., Rockville, MD (US)

(21) Appl. No.: 16/482,057

(22) PCT Filed: Jan. 30, 2018
(86) PCT No.: PCT/US2018/015910

§ 371 Date: Jul. 30, 2019

Related U.S. Application Data
(60) Provisional application No. 62/452,769, filed on Jan. 31, 2017.
(60) Provisional application No. 62/485,655, filed on Apr. 14, 2017.
(60) Provisional application No. 62/529,366, filed on Jul. 6, 2017.
(60) Provisional application No. 62/579,690, filed on Oct. 31, 2017.
(60) Provisional application No. 62/616,234, filed on Jan. 11, 2018.

(51) Int. Cl. A61K 38/47; A61P 25/28; A61K 48/00; A61K 31/436; A61K 31/573; A61K 31/365

(52) U.S. Cl. CPC A61K 38/47; A61P 25/28; A61K 48/005; C12Y 302/01076; A61K 31/436; A61K 31/573; A61K 31/365; A61K 48/0083

(57) ABSTRACT

Compositions and methods are described for the delivery of a fully human-glycosylated (HuGly) α-L-iduronidase (IDUA) to the cerebrospinal fluid of the central nervous system (CNS) of a human subject diagnosed with mucopolysaccharidosis I (MPS I).

### CROSS-REFERENCE TO RELATED APPLICATIONS

This application claims the benefit of U.S. Provisional Patent Application Nos. 62/452,769, filed Jan. 31, 2017, 62/485,655, filed Apr. 14, 2017, 62/529,366, filed Jul. 6, 2017, 62/579,690, filed Oct. 31, 2017, and 62/616,234, filed Jan. 11, 2018, which are incorporated by reference herein in their entireties.

### REFERENCE TO SEQUENCE LISTING SUBMITTED ELECTRONICALLY

This application incorporates by reference a Sequence Listing submitted with this application as text file entitled “Sequence_Listing_12656-106-228 txt” created on Jan. 16, 2018 and having a size of 80,541 bytes.

### 1. INTRODUCTION

Compositions and methods are described for the delivery of a fully human-glycosylated (HuGly) α-L-iduronidase (IDUA) to the cerebrospinal fluid of the central nervous system (CNS) of a human subject diagnosed with mucopolysaccharidosis I (MPS I).

### 2. BACKGROUND OF THE INVENTION

Mucopolysaccharidosis type I (MPS I) is a rare recessive genetic disease with an estimated incidence of 1 in 100,000 live births (Moore D et al., 2008, Orphanet Journal of Rare Diseases 3). MPS I is caused by deficiency of α-l-iduronidase (IDUA), an enzyme required for the lysosomal catabolism of the ubiquitous complex polysaccharides heparan sulfate and dermatan sulfate. These polysaccharides, called glycosaminoglycans (GAGs), accumulate in tissues of MPS I patients, resulting in characteristic storage lesions and diverse disease sequelae. Patients may exhibit short stature, bone and joint deformities, coarsened facial features, hepatosplenomegaly, cardiac valve disease, obstructive sleep apnea, recurrent upper respiratory infections, hearing impairment, carpal tunnel syndrome, and vision impairment due to corneal clouding (Beck M, et al., 2014, The natural history of MPS I: global perspectives from the MPS I Registry. Genetics in medicine: official journal of the American College of Medical Genetics 16(10):759-765). In addition, many patients develop symptoms related to GAG storage in the central nervous system, which can include hydrocephalus, spinal cord compression and, in some patients, cognitive impairment.

MPS I patients span a broad spectrum of disease severity and extent of CNS involvement. This variability in severity correlates with residual IDUA expression; patients with two mutations that result in no active enzyme expression—including nonsense mutations, deletions, and some missense mutations—typically present with symptoms before two years of age, and universally exhibit severe cognitive decline after an initial period of normal development (Terlato N J & Cox G F, 2003, Genetics in Medicine: official journal of the American College of Medical Genetics 5(4):286-294). This severe form of MPS I is also referred to as Hurler (H) syndrome. Patients with at least one mutation that results in production of a small amount of active IDUA exhibit an attenuated phenotype, referred to as Hurler-Scheie (HS) syndrome or Scheie syndrome. These patients may present with symptoms early in childhood or may not be identified until after the first decade of life. Although onset is generally later and severity may be reduced, patients with the attenuated form of MPS I can experience any of the same somatic features as those with Hurler syndrome (Vijay S & Wraith J E, 2005, Acta Paediatrica 94(7):872-877). Patients with attenuated MPS I also experience high rates of neurological complications, including spinal cord compression and hydrocephalus. Cognitive impairment is reported in approximately 30% of patients classified as having attenuated MPS I (Beck M, et al., 2014, Genetics in medicine: official journal of the American College of Medical Genetics 16(10):759-765).

Enzyme replacement therapy (ERT) [Aldurazyme® (laronidase)] has been accepted as standard of care for systemic symptoms of MPS I, but does not treat the CNS manifestations (de Ru M H, et al., 2011, Orphanet Journal of Rare Diseases 6:9; Wraith J E, et al., 2007, Pediatrics 120(1): E37-E46). Hematopoietic stem cell transplantation (HSCT) does impact the neurocognitive symptoms of MPS I, but there are important limitations of the procedure. HSCT for MPS I is associated with substantial morbidity and up to 20% mortality, and treatment is incomplete as patients still encounter neurocognitive decline up to 1 year after HSCT while IDUA expression stabilizes (de Ru M B, et al., 2011, Orphanet Journal of Rare Diseases 6:9; Fleming D R, et al., 1998, Pediatric transplantation 2(4):299-304; Boelens J J, et al., 2007, Bone Marrow Transplantation 40(3):225-233; Souillet G, et al., 2003, Bone Marrow Transplantation 31(12):1105-1117; Whitley C B, et al., 1993, American Journal of Medical Genetics 46(2):209-218). Among successfully engrafted patients, intelligence typically remains significantly below normal.

### 3. SUMMARY OF THE INVENTION

The invention involves the delivery of a fully human-glycosylated (HuGly) α-L-iduronidase (HuGlyIDUA) to the cerebrospinal fluid (CSF) of the central nervous system of a human subject diagnosed with mucopolysaccharidosis I (MPS I), including, but not limited to patients diagnosed with Hurler, Hurler-Scheie, or Scheie syndrome. In a preferred embodiment, the treatment is accomplished via gene therapy—e.g., by administering a viral vector or other DNA expression construct encoding human IDUA (hIDUA), or a derivative of hIDUA, to the CSF of a patient (human subject) diagnosed with MPS I, so that a permanent depot of transduced cells is generated that continuously supplies the fully human-glycosylated transgene product to the CNS. HuGlyIDUA secreted from the depot into the CSF will be endocytosed by cells in the CNS, resulting in “cross-correction” of the enzymatic defect in the recipient cells. In an alternative embodiment, the HuGlyIDUA can be produced in cell culture and administered as an enzyme replacement therapy (“ERT”), e.g., by injecting the enzyme. However, the gene therapy approach offers several advantages over ERT systemic delivery of the enzyme will not result in treating the CNS because the enzyme cannot cross the blood brain barrier; and, unlike the gene therapy approach of the invention, direct delivery of the enzyme to the CNS would require repeat injections which are not only burdensome, but pose a risk of infection.

The HuGlyIDUA encoded by the transgene can include, but is not limited to human IDUA (hIDUA) having the amino acid sequence of SEQ ID NO. 1 (as shown in FIG. 1), and derivatives of hIDUA having amino acid substitutions, deletions, or additions, e.g., including but not limited to amino acid substitutions selected from corresponding non-conserved residues in orthologs of IDUA shown in FIG. 2, with the proviso that such mutations do not include any that have been identified in severe, severe-intermediate, intermediate, or attenuated MPS I phenotypes shown in FIG. 3 (from, Saito et al., 2014, Mol Genet Metab 111:107-112, Table 1 listing 57 MPS I mutations, which is incorporated by reference herein in its entirety); or reported by Venturi et al., 2002, Human Mutation #522 Online (“Venturi 2002”), or Bertola et al., 2011 Human Mutation 32:E2189-E2210 (“Bertola 2011”), each of which is incorporated by reference herein in its entirety.

For example, amino acid substitutions at a particular position of hIDUA can be selected from among corresponding non-conserved amino acid residues found at that position in the IDUA orthologs depicted in FIG. 2 (showing alignment of orthologs as reported Maita et al., 2013, PNAS 110:14628, FIG. S8 which is incorporated by reference herein in its entirety), with the proviso that such substitutions do not include any of the deleterious mutations shown in FIG. 3 or reported in Venturi 2002 or Bertola 2011 supra. The resulting transgene product can be tested using conventional assays in vitro, in cell culture or test animals to ensure that the mutation does not disrupt IDUA function. Preferred amino acid substitutions, deletions or additions selected should be those that maintain or increase enzyme activity, stability or half-life of IDUA, as tested by conventional assays in vitro, in cell culture or animal models for MPS I. For example, the enzyme activity of the transgene product can be assessed using a conventional enzyme assay with 4-methylumbelliferyl α-L-iduronide as the substrate (see, e.g., Hopwood et al., 1979, Clin Chim Acta 92: 257-265; Clements et al., 1985, Eur J Biochem 152: 21-28; and Kakkis et al., 1994, Prot Exp Purif 5: 225-232 for exemplary IDUA enzyme assays that can be used, each of which is incorporated by reference herein in its entirety). The ability of the transgene product to correct MPS I phenotype can be assessed in cell culture; e.g., by transducing MPS I cells in culture with a viral vector or other DNA expression construct encoding hIDUA or a derivative; by adding the rHuGlyIDUA or a derivative to MPS I cells in culture; or by co-culturing MPS I cells with human host cells engineered to express and secrete rHuGlyIDUA or a derivative, and determining correction of the defect in the MPS I cultured cells, e.g., by detecting IDUA enzyme activity and/or reduction in GAG storage in the MPS I cells in culture (see e.g., Myerowitz & Neufeld, 1981, J Biol Chem 256: 3044-3048; and Anson et al. 1992, Hum Gene Ther 3: 371-379, each of which is incorporated by reference herein in its entirety).

Animal models for MPS I have been described for mice (see, e.g., Clarke et al., 1997, Hum Mol Genet 6(4):503-511), the domestic shorthair cat (see, e.g., Haskins et al., 1979, Pediatr Res 13(11):1294-97), and several breeds of dog (see, e.g., Menon et al., 1992, Genomics 14(3):763-768; Shull et al., 1982, Am J Pathol 109(2):244-248). The MPS I model in dog resembles Hurler syndrome, the most severe form of MPS I, since the IDUA mutation results in no detectable protein. High gene homology between IDUA proteins (see alignment in FIG. 2) means that hIDUA is functional in animals, and treatments encompassing hIDUA may be tested on these animal models.

Preferably, the rHuGlyIDUA transgene should be controlled by expression control elements that function in neurons and/or glial cells, e.g., the CB7 promoter (a chicken β-actin promoter and CMV enhancer), and can include other expression control elements that enhance expression of the transgene driven by the vector (e.g., chicken β-actin intron and rabbit β-globin poly A signal). The cDNA construct for the hIDUA transgene should include a coding sequence for a signal peptide that ensures proper co- and post-translational processing (glycosylation and protein sulfation) by the transduced CNS cells. Such signal peptides used by CNS cells may include but are not limited to:

Oligodendrocyte-myelin glycoprotein (hOMG)
signal peptide:
(SEQ ID NO: 2)
MEYQILKMSLCLFILLFLTPGILC
Cellular repressor of E1A-stimulated genes
2 (hCREG2) signal peptide:
(SEQ ID NO: 3)
MSVRRGRRPARPGTRLSWLLCCSALLSPAAG
V-set and transmembrane domain containing
2B (hVSTM2B) signal peptide:
(SEQ ID NO: 4)
MEQRNRLGALGYLPPLLLHALLLFVADA
Protocadherin alpha-1 (hPCADHA1) signal peptide:
(SEQ ID NO: 5)
MVFSRRGGLGARDLLLWLLLLAAWEVGSG
FAM19A1 (TAFA1) signal peptide:
(SEQ ID NO: 6)
MAMVSAMSWVLYLWISACA
Interleukin-2 signal peptide:
(SEQ ID NO: 14)
MYRMQLLSCIALILALVTNS

Signal peptides may also be referred to herein as leader sequences or leader peptides.

The recombinant vector used for delivering the transgene should have a tropism for cells in the CNS, including but limited to neurons and/or glial cells. Such vectors can include non-replicating recombinant adeno-associated virus vectors (“rAAV”), particularly those bearing an AAV9 or AAVrh10 capsid are preferred. AAV variant capsids can be used, including but not limited to those described by Wilson in U.S. Pat. No. 7,906,111 which is incorporated by reference herein in its entirety, with AAV/hu.31 and AAV/hu.32 being particularly preferred; as well as AAV variant capsids described by Chatterjee in U.S. Pat. No. 8,628,966, 8,927,514 and Smith et al., 2014, Mol Ther 22: 1625-1634, each of which is incorporated by reference herein in its entirety. However, other viral vectors may be used, including but not limited to lentiviral vectors, vaccinia viral vectors, or non-viral expression vectors referred to as “naked DNA” constructs (see Section 5.2).

Pharmaceutical compositions suitable for administration to the CSF comprise a suspension of the rHuGlyIDUA vector in a formulation buffer comprising a physiologically compatible aqueous buffer, a surfactant and optional excipients. In certain embodiments, the pharmaceutical compositions are suitable for intrathecal administration. In certain embodiments, the pharmaceutical compositions are suitable for intracisternal administration (injection into the cisterna magna). In certain embodiments, the pharmaceutical compositions are suitable for injection into the subarachnoid space via a C1-2 puncture. In certain embodiments, the pharmaceutical compositions are suitable for intracerebroventricular administration. In certain embodiments, the pharmaceutical compositions are suitable for administration via lumbar puncture.

Therapeutically effective doses of the recombinant vector should be administered to the CSF via intrathecal administration (i.e., injection into the subarachnoid space so that the recombinant vectors distribute through the CSF and transduce cells in the CNS). This can be accomplished in a number of ways—e.g., by intracranial (cisternal or ventricular) injection, or injection into the lumbar cistern. For example intracisternal (IC) injection (into the cisterna magna) can be performed by CT-guided suboccipital puncture; or injection into the subarachnoid space can be performed via a C1-2 puncture when feasible for the patient; or lumbar puncture (typically diagnostic procedures performed in order to collect a sample of CSF) can be used to access the CSF. Alternatively, intracerebroventricular (ICV) administration (a more invasive technique used for the introduction of antiinfective or anticancer drugs that do not penetrate the blood-brain barrier) can be used to instill the recombinant vectors directly into the ventricles of the brain. Alternatively, intranasal administration may be used to deliver the recombinant vector to the CNS. Doses that maintain a CSF concentration of rHuGlyIDUA at a Cₘᵢₙ of at least 9.25 μg/mL or concentrations ranging from 9.25 to 277 μg/mL should be used.

CSF concentrations can be monitored by directly measuring the concentration of rHuGlyIDUA in the CSF fluid obtained from occipital or lumbar punctures, or estimated by extrapolation from concentrations of the rHuGlyIDUA detected in the patient's serum. In certain embodiments, 10 ng/mL to 100 ng/mL of rHuGlyIDUA in the serum is indicative of 1 to 30 mg of rHuGlyIDUA in the CSF. In certain embodiments, the recombinant vector is administered to the CSF at a dose that maintains 10 ng/mL to 100 ng/mL of rHuGlyIDUA in the serum.

By way of background, human IDUA is translated as a 653 amino acid polypeptide and is N-glycosylated at six potential sites (N110, N190, N336, N372, N415 and N451) depicted in FIG. 1. The signal sequence is removed and the polypeptide is processed into the mature form in lysosomes: a 75 kDa intracellular precursor is trimmed to 72 kDa in several hours, and eventually, over 4 to 5 days, is processed to a 66 kDa intracellular form. A secreted form of IDUA (76 kDa or 82 kDa depending on the assay used) is readily endocytosed by cells via the mannose-6-phosphate receptor and similarly processed to the smaller intracellular forms. (See, Myerowitz & Neufeld, 1981, J Biol Chem 256: 3044-3048; Clements et al., 1989, Biochem J. 259: 199-208; Taylor et al., 1991, Biochem J. 274: 263-268; and Zhao et al., 1997 J Biol Chem 272:22758-22765 each of which is incorporated by reference herein in its entirety).

The overall structure of hIDUA consists of three domains: residues 42-396 form a classic (β/α) triosephosphate isomerase (TIM) barrel domain; residues 27-42 and 397-545 form a β-sandwich domain with a short helix-loop-helix (482-508); and residues 546-642 form an Ig-like domain. The latter two domains are linked through a disulfide bridge between C⁵⁴¹ and C⁵⁷⁷. The β-sandwich and Ig-like domains are attached to the first, seventh, and eighth α-helices of the TIM barrel. A β-hairpin (β12(β13) is inserted between the eighth β-strand and the eighth α-helix of the TIM barrel, which includes N-glycosylated N³⁷² which is required for substrate binding and enzymatic activity. (See, FIG. 1 and crystal structure described in Maita et al., 2013, PNAS 110: 14628-14633, and Saito et al., 2014, Mol Genet Metab 111: 107-112 each of which is incorporated by reference herein in its entirety).

The invention is based, in part, on the following principles:
(i) Neuron and glial cells in the CNS are secretory cells that possess the cellular machinery for post-translational processing of secreted proteins—including glycosylation and tyrosine-O-sulfation—robust processes in the CNS. See, e.g., Sleat et al., 2005, Proteomics 5: 1520-1532, and Sleat 1996, J Biol Chem 271: 19191-98 which describes the human brain mannose-6-phosphate (M6P) glycoproteome and notes that the brain contains more proteins with a much greater number of individual isoforms and mannose-6-phosphorylated proteins than found in other tissues; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by neuronal cells, each of which is incorporated by reference in its entirety for post-translational modifications made by human CNS cells.
(ii) hIDUA has six asparaginal (“N”) glycosylation sites identified in FIG. 1 (N¹¹⁰FT; N¹⁹⁰VS; N³³⁶TT; N³⁷²NT; N⁴¹⁵HT; N⁴⁵¹RS). N-glycosylation of N³⁷² is required for binding to substrate and enzymatic activity, and mannose-6-phosphorylation is required for cellular uptake of the secreted enzyme and cross-correction of MPS I cells. The N-linked glycosylation sites contain complex, high mannose and phosphorylated mannose carbohydrate moieties (FIG. 4), but only the secreted form is taken up by cells. (Myerowitz & Neufeld, 1981, supra). The gene therapy approach described herein should result in the continuous secretion of an IDUA glycoprotein of 76-82 kDa as measured by polyacrylamide gel electrophoresis (depending on the assay used) that is 2,6-sialylated and mannose-6-phosphorylated. The secreted glycosylated/phosphorylated IDUA should be taken up and correctly processed by untransduced neural and glial cells in the CNS.
(iii) The cellular and subcellular trafficking/uptake of lysosomal proteins is through M6P. It is possible to measure the M6P content of a secreted protein, as done in Daniele 2002 (Biochimica et Biophysica Acta 1588(3):203-9) for the iduronate-2-sulfatase enzyme. In the presence of inhibitory M6P (e.g., 5 mM), the uptake of the enzyme precursor generated by non-neuronal or non-glial cells, such as the genetically engineered kidney cells of Daniele 2002, is predicted to decrease to levels close to that of the control cells, as was shown in Daniele 2002. While in the presence of inhibitory M6P, the uptake of enzyme precursor generated by brain cells, such as neuronal and glial cells, is predicted to remain at a high level, as was shown in Daniele 2002, where the uptake was four times higher than control cells and comparable to the level of enzyme activity (or uptake) of enzyme precursor generated by genetically engineered kidney cells without the presence of inhibitory M6P. This assay allows for a way to predict the M6P content in an enzyme precursor generated by brain cells, and, in particular, to compare the M6P content in enzyme precursors generated by different types of cells. The gene therapy approach described herein should result in the continuous secretion of hIDUA that may be taken up into neuronal and glial cells at a high level in the presence of inhibitory M6P in such an assay.
(iv) In addition to the N-linked glycosylation sites, hIDUA contains a tyrosine (“Y”) sulfation site (ADTPIY²⁹⁶NDEADPLVGWS) near the domain containing N³⁷² required for binding and activity. (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). While not intending to be bound by any theory, sulfation of this site in hIDUA may be critical to activity since mutations within the tyrosine-sulfation region (e.g., W306L) are known to be associated with decreased enzymatic activity and disease. (See, Maita et al., 2013, PNAS 110:14628 at pp. 14632-14633).
(v) The glycosylation of hIDUA by human cells of the CNS will result in the addition of glycans that can improve stability, half-life and reduce unwanted aggregation of the transgene product. Significantly, the glycans that are added to HuGlyIDUA of the invention are highly processed complex-type biantennary N-glycans that include 2,6-sialic acid, incorporating Neu5Ac (“NANA”) but not its hydroxylated derivative, NeuGc (N-Glycolylneuraminic acid, i.e., “NGNA” or “Neu5Gc”). Such glycans are not present in laronidase which is made in CHO cells that do not have the 2,6-sialyltransferase required to make this post-translational modification, nor do CHO cells produce bisecting GlcNAc, although they do add Neu5Gc (NGNA) as sialic acid not typical (and potentially immunogenic) to humans instead of Neu5Ac (NANA). See, e.g., Dumont et al., 2016, Critical Rev in Biotech 36(6):1110-1122 (Early Online pp. 1-13 at p. 5); and Hague et al., 1998 Electrophor 19:2612-2630 (“[t]he CHO cell line is considered ‘phenotypically restricted,’ in terms of glycosylation, due to the lack of an α2,6-sialyl-transferase”). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuGlyIDUA of the invention should reduce immunogenicity of the transgene product and improve efficacy.
(vi) Tyrosine-sulfation of hIDUA—a robust post-translational process in human CNS cells should result in improved processing and activity of transgene products. The significance of tyrosine-sulfation of lysosomal proteins has not been elucidated; but in other proteins it has been shown to increase avidity of protein-protein interactions (antibodies and receptors), and to promote proteolytic processing (peptide hormone). (See, Moore, 2003, J Biol. Chem. 278:24243-46; and Bundegaard et al., 1995, The EMBO J 14: 3073-79). The tyrosylprotein sulfotransferase (TPST1) responsible for tyrosine-sulfation (which may occur as a final step in IDUA processing) is apparently expressed at higher levels (based on mRNA) in the brain (gene expression data for TPST1 may be found, for example, at the EMBL-EBI Expression Atlas, accessible at http://www.ebi.ac.uk/gxa/home). Such post-translational modification, at best, is under-represented in laronidase—a CHO cell product. Unlike human CNS cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).
(vii) Immunogenicity of a transgene product could be induced by various factors, including the immune condition of the patient, the structure and characteristics of the infused protein drug, the administration route, and the duration of treatment. Process-related impurities, such as host cell protein (HCP), host cell DNA, and chemical residuals, and product-related impurities, such as protein degradants and structural characteristics, such as glycosylation, oxidation and aggregation (sub-visible particles), may also increase immunogenicity by serving as an adjuvant that enhances the immune response. The amounts of process-related and product-related impurities can be affected by the manufacturing process: cell culture, purification, formulation, storage and handling, which can affect commercially manufactured IDUA products. In gene therapy, proteins are produced in vivo, such that process-related impurities are not present and protein products are not likely to contain product-related impurities/degradants associated with proteins produced by recombinant technologies, such as protein aggregation and protein oxidation. Aggregation, for example, is associated with protein production and storage due to high protein concentration, surface interaction with manufacturing equipment and containers, and the purification process with certain buffer systems. But these conditions that promote aggregation are not present when a transgene is expressed in vivo. Oxidation, such as methionine, tryptophan and histidine oxidation, is also associated with protein production and storage, caused, for example, by stressed cell culture conditions, metal and air contact, and impurities in buffers and excipients. The proteins expressed in vivo may also oxidize in a stressed condition, but humans, like many organisms, are equipped with an antioxidation defense system, which not only reduces the oxidation stress, but can also repairs and/or reverses the oxidation. Thus, proteins produced in vivo are not likely to be in an oxidized form. Both aggregation and oxidation could affect the potency, PK (clearance) and can increase immunogenicity concerns. The gene therapy approach described herein should result in the continuous secretion of hIDUA with a reduced immunogenicity compared to commercially manufactured products.

For the foregoing reasons, the production of HuGlyIDUA should result in a “biobetter” molecule for the treatment of MPS I accomplished via gene therapy e.g., by administering a viral vector or other DNA expression construct encoding HuGlyIDUA to the CSF of a patient (human subject) diagnosed with an MPS I disease (including but not limited to Hurler, Hurler-Scheie, or Scheie) to create a permanent depot in the CNS that continuously supplies a fully human-glycosylated, mannose-6-phosphorylated, sulfated transgene product secreted by the transduced CNS cells. The HuGlyIDUA transgene product secreted from the depot into the CSF will be endocytosed by cells in the CNS, resulting in “cross-correction” of the enzymatic defect in the MPS I recipient cells.

It is not essential that every hIDUA molecule produced either in the gene therapy or protein therapy approach be fully glycosylated and sulfated. Rather, the population of glycoproteins produced should have sufficient glycosylation (including 2,6-sialylation and mannose-6-phophorylation) and sulfation to demonstrate efficacy. The goal of gene therapy treatment of the invention is to slow or arrest the progression of disease. Efficacy may be monitored by measuring cognitive function (e.g., prevention or decrease in neurocognitive decline); reductions in biomarkers of disease (such as GAG) in CSF and or serum; and/or increase in IDUA enzyme activity in CSF and/or serum. Signs of inflammation and other safety events may also be monitored.

As an alternative, or an additional treatment to gene therapy, the rHuGlyIDUA glycoprotein can be produced in human cell lines by recombinant DNA technology and the glycoprotein can be administered to patients diagnosed with MPS I systemically and/or into the CSF for ERT). Human cell lines that can be used for such recombinant glycoprotein production include but are not limited to HT-22, SK-N-MC, HCN-1A, HCN-2, NT2, SH-SY5y, hNSC11, ReNcell VM, human embryonic kidney 293 cells (HEK293), fibrosarcoma HT-1080, HKB-11, CAP, HuH-7, and retinal cell lines, PER.C6, or RPE to name a few (see, e.g., Dumont et al., 2016, Critical Rev in Biotech 36(6):1110-1122 “Human cell lines for biopharmaceutical manufacturing: history, status, and future perspectives” which is incorporated by reference in its entirety for a review of the human cell lines that could be used for the recombinant production of the rHuGlyIDUA glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation.

While the delivery of rHuGlyIDUA should minimize immune reactions, the clearest potential source of toxicity related to CNS-directed gene therapy is generating immunity against the expressed hIDUA protein in human subjects who are genetically deficient for IDUA and, therefore, potentially not tolerant of the protein and/or the vector used to deliver the transgene.

Thus, in a preferred embodiment, it is advisable to co-treat the patient with immune suppression therapy—especially when treating patients with severe disease who have close to zero levels of IDUA (e.g., Hurler). Immune suppression therapies involving a regimen of tacrolimus or rapamycin (sirolimus), for example, in combination with mycophenolic acid or in combination with a corticosteroid such as prednisolone and/or methylprednisolone, or other immune suppression regimens used in tissue transplantation procedures can be employed. Such immune suppression treatment may be administered during the course of gene therapy, and in certain embodiments, pre-treatment with immune suppression therapy may be preferred. Immune suppression therapy can be continued subsequent to the gene therapy treatment, based on the judgment of the treating physician, and may thereafter be withdrawn when immune tolerance is induced; e.g., after 180 days.

Combinations of delivery of the HuGlyIDUA to the CSF accompanied by delivery of other available treatments are encompassed by the methods of the invention. The additional treatments may be administered before, concurrently or subsequent to the gene therapy treatment. Available treatments for MPS I that could be combined with the gene therapy of the invention include but are not limited to enzyme replacement therapy using laronidase administered systemically or to the CSF; and/or HSCT therapy.

### ILLUSTRATIVE EMBODIMENTS

1. A method for treating a human subject diagnosed with mucopolysaccharidosis I (MPS I), comprising delivering to the cerebrospinal fluid of the brain of said human subject a therapeutically effective amount of recombinant human α-L-iduronidase (IDUA) produced by human neuronal cells.

2. A method for treating a human subject diagnosed with MPS I, comprising delivering to the cerebrospinal fluid of the brain of said human subject a therapeutically effective amount of recombinant human IDUA produced by human glial cells.

3. The method of paragraph 1 or 2, further comprising administering an immune suppression therapy to said subject before or concurrently with the human IDUA treatment and continuing immune suppression therapy thereafter.

4. A method of treating a human subject diagnosed with MPS I, comprising:

delivering to the cerebrospinal fluid of the brain of said human subject, a therapeutically effective amount of a α2,6-sialylated human IDUA.

5. A method of treating a human subject diagnosed with mucopolysaccharidosis I (MPS I), comprising:

delivering to the cerebrospinal fluid of the brain of said human subject, a therapeutically effective amount of a glycosylated human IDUA that does not contain detectable NeuGc.

6. A method of treating a human subject diagnosed with mucopolysaccharidosis I (MPS I), comprising:

delivering to the cerebrospinal fluid of the brain of said human subject, a therapeutically effective amount of a glycosylated human IDUA that does not contain detectable NeuGc and/or α-Gal antigen; and

administering an immune suppression therapy to said subject before or concurrently with the human IDUA treatment and continuing immune suppression therapy thereafter.

7. A method of treating a human subject diagnosed with mucopolysaccharidosis I (MPS I), comprising:

delivering to the cerebrospinal fluid of the brain of said human subject, a therapeutically effective amount of human IDUA that contains tyrosine-sulfation.

8. A method of treating a human subject diagnosed with mucopolysaccharidosis I (MPS I), comprising administering to the brain of said human subject an expression vector encoding human IDUA, wherein said IDUA is α2,6-sialylated upon expression from said expression vector in a human, immortalized neuronal cell.

9. A method of treating a human subject diagnosed with mucopolysaccharidosis I (MPS I), comprising administering to the brain of said human subject an expression vector encoding human IDUA, wherein said IDUA is glycosylated but does not contain detectable NeuGc upon expression from said expression vector in a human, immortalized neuronal cell.

10. A method of treating a human subject diagnosed with mucopolysaccharidosis I (MPS I), comprising administering to the brain of said human subject an expression vector encoding human IDUA, wherein said IDUA is glycosylated but does not contain detectable NeuGc and/or α-Gal antigen upon expression from said expression vector in a human, immortalized neuronal cell.

11. A method of treating a human subject diagnosed with mucopolysaccharidosis I (MPS I), comprising administering to the brain of said human subject an expression vector encoding human IDUA, wherein said IDUA is tyrosine-sulfated upon expression from said expression vector in a human, immortalized neuronal cell.

12. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases said IDUA containing a α2,6-sialylated glycan.

13. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc.

14. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc and/or α-Gal antigen.

15. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases said IDUA containing a tyrosine-sulfation.

16. The method of any one of paragraphs 3 to 15 further comprising administering an immune suppression therapy to said subject, comprising administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment and continuing thereafter.

17. The method of paragraph 16 in which the immune suppression therapy is withdrawn after 180 days.

18. The method of any one of paragraphs 1 to 17 in which the human IDUA comprises the amino acid sequence of SEQ ID NO. 1.

19. The method of paragraph 18 further comprising administering an immune suppression therapy to said subject, comprising administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment.

20. The method of paragraph 19 in which the immune suppression therapy is withdrawn after 180 days.

21. The method of paragraph 12 in which production of said IDUA containing a α2,6-sialylated glycan is confirmed by transducing a human neuronal cell line with said recombinant nucleotide expression vector in cell culture.

22. The method of paragraph 13 in which production of said glycosylated IDUA that does not contain detectable NeuGc is confirmed by transducing a human neuronal cell line with said recombinant nucleotide expression vector in cell culture.

23. The method of any one of paragraph 14 in which production of said glycosylated IDUA that does not contain detectable NeuGc and/or α-Gal antigen is confirmed by transducing a human neuronal cell line with said recombinant nucleotide expression vector in cell culture.

24. The method of paragraph 15 in which production of said IDUA containing a tyrosine-sulfation is confirmed by transducing a human neuronal cell line with said recombinant nucleotide expression vector in cell culture.

25. The method of any one of paragraphs 21-24, in which production is confirmed in the presence and absence of mannose-6-phosphate.

26. The method of any one of paragraphs 8-15 and 21-25, or of any one of paragraphs 16-17 when dependent directly or indirectly on any one of claims 8-15, wherein the expression vector or recombinant nucleotide expression vector encodes a signal peptide.

27. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases said IDUA containing a α2,6-sialylated glycan;

wherein said recombinant vector, when used to transduce human neuronal cells in culture results in production of said IDUA containing a α2,6-sialylated glycan in said cell culture.

28. A method of treating a human subject diagnosed with mucopolysaccharidosis I (MPS I), comprising: administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc; wherein said recombinant vector, when used to transduce human neuronal cells in culture results in production of said IDUA that is glycosylated but does not contain detectable NeuGc in said cell culture.

29. A method of treating a human subject diagnosed with mucopolysaccharidosis I (MPS I), comprising: administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc and/or α-Gal antigen; wherein said recombinant vector, when used to transduce human neuronal cells in culture results in production of said IDUA that is glycosylated but does not contain detectable NeuGc and/or α-Gal antigen in said cell culture.

30. A method of treating a human subject diagnosed with mucopolysaccharidosis I (MPS I), comprising:

administering to the cerebrospinal fluid of the brain of said human subject, a therapeutically effective amount of a recombinant nucleotide expression vector encoding human IDUA, so that a depot is formed that releases said IDUA that contains a tyrosine-sulfation;

wherein said recombinant vector, when used to transduce human neuronal cells in culture results in production of said IDUA that is tyrosine-sulfated in said cell culture.

31. The method of any of paragraphs 27 to 30 further comprising administering an immune suppression therapy to said subject, comprising administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment and continuing thereafter.

32. The method of paragraph 31 in which the immune suppression therapy is withdrawn after 180 days.

33. The method of any one of paragraphs 1-32, wherein the human subject is younger than 3 years of age.

34. The method of any one of paragraphs 8-15 and 21-33, or of any one of paragraphs 16-20 when dependent directly or indirectly on any one of claims 8-15, wherein the human subject is younger than 3 years of age and the expression vector or the recombinant nucleotide expression vector is administered (for example, IC administration (such as by suboccipital injection)) at a dose of 1×10¹⁰ GC/g brain mass or 5×10¹⁰ GC/g brain mass (for example, as a single flat dose).

35. The method of any one of paragraphs 8-15 and 21-33, or of any one of paragraphs 16-20 when dependent directly or indirectly on any one of claims 8-15, wherein the human subject is younger than 3 years of age and the expression vector or the recombinant nucleotide expression vector is administered (for example, IC administration (such as by suboccipital injection)) at a dose ranging from 1×10¹⁰ GC/g brain mass to 5×10¹⁰ GC/g brain mass (for example, as a single flat dose).

### 4. BRIEF DESCRIPTION OF THE DRAWINGS

FIG. 1. The amino acid sequence of human IDUA. Six N-linked glycosylation sites (N) are bold and underlined; one tyrosine-O-sulfation site (Y) is bold and underlined, and the full sulfation site sequence (ADTPIYNDEADPLVGWS) is shaded; and a disulfide bond (two cysteine residues; C) is bold and underlined. The N-terminus of the secreted recombinant product made in CHO cells is A²⁶, whereas the N-terminus of the native intracellular enzyme of human liver is E²⁷ (See, Kakkis et al., 1994, Prot Exp Purif 5: 225-232, at p. 230).

FIG. 2. Multiple sequence alignment of hIDUA with known orthologs. The sequences were aligned using Clustal X ver.2 (Larkin M A, et al., 2007, Clustal W and Clustal X version 2.0. Bioinformatics 23(21):2947-2948). The names of the species and protein IDs are as follows: human (Homo sapiens; NP_000194.2), dog (Canis familiaris; M81893.1), cow (Bos taurus; XP_002688492.1), mouse (Mus musculus; NP_032351.2), rat (Rattus norvegicus; NP_001165555.1), platypus (Ornithorhynchus anatinus; XP 001514102.2), chicken (Gallus gallus; NP_001026604.1), Xenopus (Xenopus laevis; NP_001087031.1), zebrafish (Danio rerio; XP 001923689.3), sea urchin (Strongylocentrotus purpuratus; XP 796813.3) ciona (Ciona intestinalis; XP 002120937.1), and fruit fly (Drosophila melanogaster; NP_609489.1). The N-glycosylation site in the human protein (N110, N190, N336, N372, T374, N415, N451); the residues involved in substrate binding (R89, H91, N181, E182, H262, K264, E299, D349, and R363) and the interaction with the N-glycan at N372 (P54, H58, W306, S307, Y355, R368, and Q370) are indicated by shading. (Adapted from: Maita et al., 2013, PNAS 110: 14628-14633; Supplementary Material, FIG. S8).

FIG. 3. MPS I mutations, structural changes in IDUA and phenotypes. (From Saito et al., Mol Genet Metab 111:107-112, Table 1).

FIG. 4. Oligosaccharides at the six glycosylation sites of recombinant human O-L-iduronidase secreted by CHO cells. C, complex; M, high mannose; P, phosphorylated high mannose. Capital letters denote well identified, major oligosaccharides, whereas lowercase letters denote minor or incompletely characterized components. (From, Zhao et al., 1997, J Biol Chem 272: 22758-22765).

FIG. 5. Clustal Multiple Sequence Alignment of AAV capsids 1-9 (SEQ ID NOs: 16-26). Amino acid substitutions (shown in bold in the bottom rows) can be made to AAV9 and AAV8 capsids by “recruiting” amino acid residues from the corresponding position of other aligned AAV capsids. Sequence regions designated by “HVR”=hypervariable regions.

### 5. DETAILED DESCRIPTION OF THE INVENTION

The invention involves the delivery of a fully human-glycosylated (HuGly) α-L-iduronidase (HuGlyIDUA) to the cerebrospinal fluid (CSF) of the central nervous system of a human subject diagnosed with mucopolysaccharidosis I (MPS I), including, but not limited to patients diagnosed with Hurler, Hurler-Scheie, or Scheie syndrome. In a preferred embodiment, the treatment is accomplished via gene therapy—e.g., by administering a viral vector or other DNA expression construct encoding human IDUA (hIDUA), or a derivative of hIDUA, to the CSF of a patient (human subject) diagnosed with MPS I, so that a permanent depot of transduced cells is generated that continuously supplies the fully human-glycosylated transgene product to the CNS. HuGlyIDUA secreted from the depot into the CSF will be endocytosed by cells in the CNS, resulting in “cross-correction” of the enzymatic defect in the recipient cells. In an alternative embodiment, the HuGlyIDUA can be produced in cell culture and administered as an enzyme replacement therapy (“ERT”), e.g., by injecting the enzyme. However, the gene therapy approach offers several advantages over ERT—systemic delivery of the enzyme will not result in treating the CNS because the enzyme cannot cross the blood brain barrier; and, unlike the gene therapy approach of the invention, direct delivery of the enzyme to the CNS would require repeat injections which are not only burdensome, but pose a risk of infection.

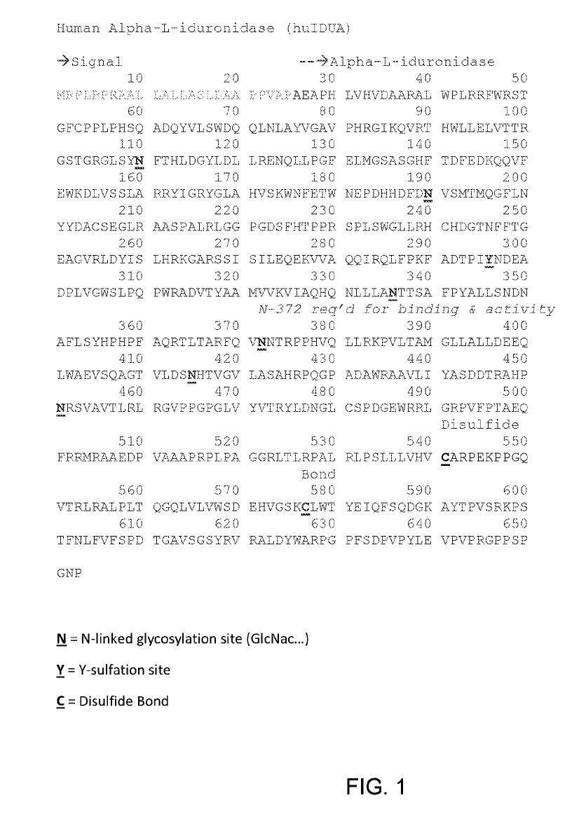

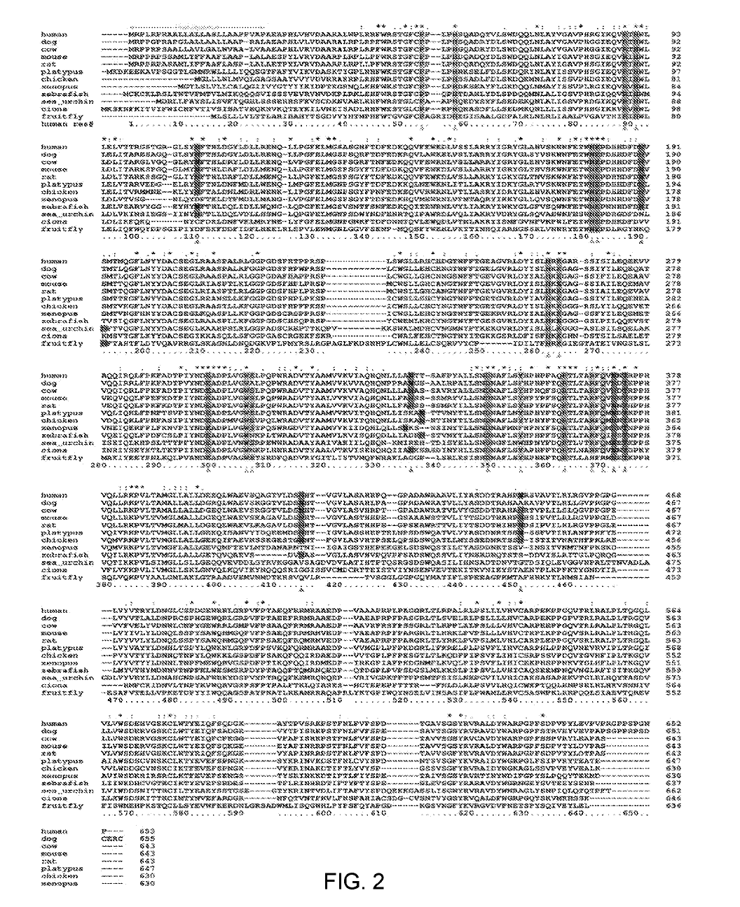

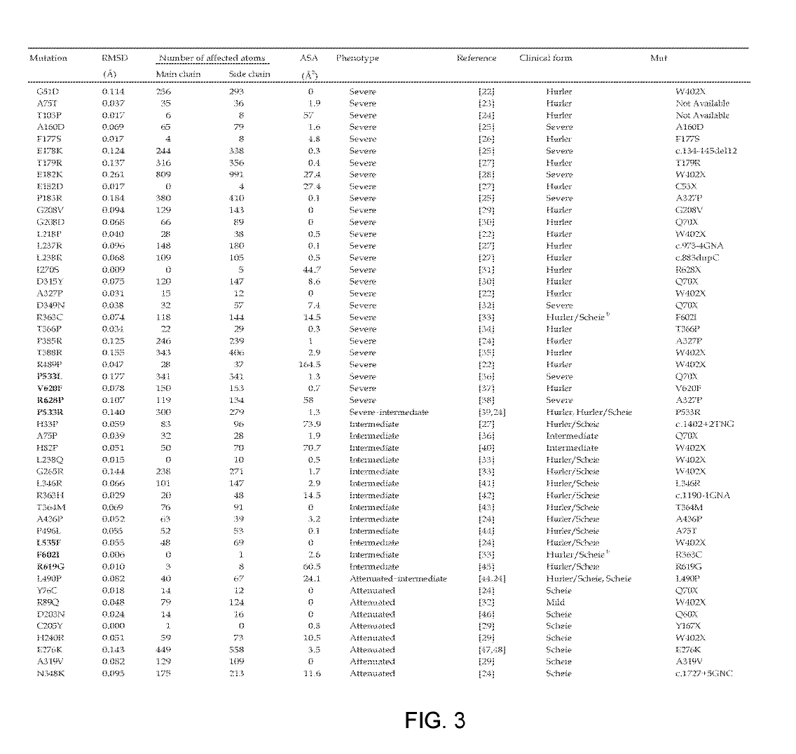

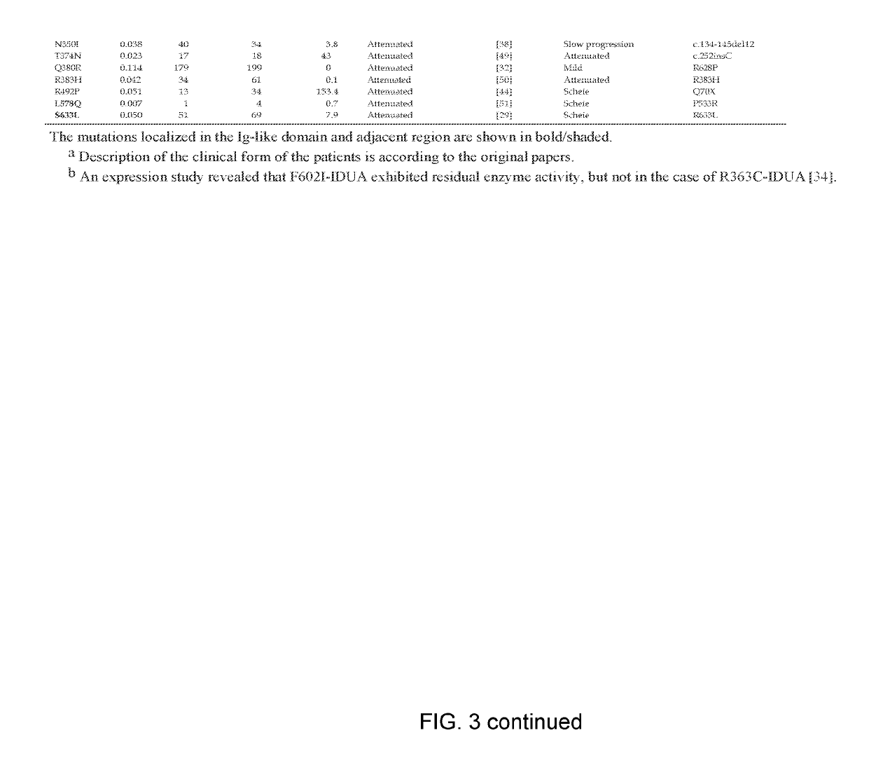

The HuGlyIDUA encoded by the transgene can include, but is not limited to human IDUA (hIDUA) having the amino acid sequence of SEQ ID NO. 1 (as shown in FIG. 1), and derivatives of hIDUA having amino acid substitutions, deletions, or additions, e.g., including but not limited to amino acid substitutions selected from corresponding non-conserved residues in orthologs of IDUA shown in FIG. 2, with the proviso that such mutations do not include any that have been identified in severe, severe-intermediate, intermediate, or attenuated MPS I phenotypes shown in FIG. 3 (from, Saito et al., 2014, Mol Genet Metab 111:107-112, Table 1 listing 57 MPS I mutations, which is incorporated by reference herein in its entirety); or reported by Venturi et al., 2002, Human Mutation #522 Online (“Venturi 2002”), or Bertola et al., 2011 Human Mutation 32:E2189-E2210 (“Bertola 2011”), each of which is incorporated by reference herein in its entirety.

For example, amino acid substitutions at a particular position of hIDUA can be selected from among corresponding non-conserved amino acid residues found at that position in the IDUA orthologs depicted in FIG. 2 (showing alignment of orthologs as reported Maita et al., 2013, PNAS 110:14628, FIG. S8 which is incorporated by reference herein in its entirety), with the proviso that such substitutions do not include any of the deleterious mutations shown in FIG. 3 or reported in Venturi 2002 or Bertola 2011 supra. The resulting transgene product can be tested using conventional assays in vitro, in cell culture or test animals to ensure that the mutation does not disrupt IDUA function. Preferred amino acid substitutions, deletions or additions selected should be those that maintain or increase enzyme activity, stability or half-life of IDUA, as tested by conventional assays in vitro, in cell culture or animal models for MPS I. For example, the enzyme activity of the transgene product can be assessed using a conventional enzyme assay with 4-methylumbelliferyl α-L-iduronide as the substrate (see, e.g., Hopwood et al., 1979, Clin Chim Acta 92: 257-265; Clements et al., 1985, Eur J Biochem 152: 21-28; and Kakkis et al., 1994, Prot Exp Purif 5: 225-232 for exemplary IDUA enzyme assays that can be used, each of which is incorporated by reference herein in its entirety). The ability of the transgene product to correct MPS I phenotype can be assessed in cell culture; e.g., by transducing MPS I cells in culture with a viral vector or other DNA expression construct encoding hIDUA or a derivative; by adding the rHuGlyIDUA or a derivative to MPS I cells in culture; or by co-culturing MPS I cells with human host cells engineered to express and secrete rHuGlyIDUA or a derivative, and determining correction of the defect in the MPS I cultured cells, e.g., by detecting IDUA enzyme activity and/or reduction in GAG storage in the MPS I cells in culture (see e.g., Myerowitz & Neufeld, 1981, J Biol Chem 256: 3044-3048; and Anson et al. 1992, Hum Gene Ther 3: 371-379, each of which is incorporated by reference herein in its entirety).

Animal models for MPS I have been described for mice (see, e.g., Clarke et al., 1997, Hum Mol Genet 6(4):503-511), the domestic shorthair cat (see, e.g., Haskins et al., 1979, Pediatr Res 13(11):1294-97), and several breeds of dog (see, e.g., Menon et al., 1992, Genomics 14(3):763-768; Shull et al., 1982, Am J Pathol 109(2):244-248). The MPS I model in dog resembles Hurler syndrome, the most severe form of MPS I, since the IDUA mutation results in no detectable protein. High gene homology between IDUA proteins (see alignment in FIG. 2) means that hIDUA is functional in animals, and treatments encompassing hIDUA may be tested on these animal models.

Preferably, the rHuGlyIDUA transgene should be controlled by expression control elements that function in neurons and/or glial cells, e.g., the CB7 promoter (a chicken β-actin promoter and CMV enhancer), and can include other expression control elements that enhance expression of the transgene driven by the vector (e.g., chicken β-actin intron and rabbit β-globin poly A signal). The cDNA construct for the huIDUA transgene should include a coding sequence for a signal peptide that ensures proper co- and post-translational processing (glycosylation and protein sulfation) by the transduced CNS cells. Such signal peptides used by CNS cells may include but are not limited to:

Oligodendrocyte-myelin glycoprotein (hOMG)
signal peptide:
(SEQ ID NO: 2)
MEYQILKMSLCLFILLFLTPGILC
Cellular repressor of E1A-stimulated genes
2 (hCREG2) signal peptide:
(SEQ ID NO: 3)
MSVRRGRRPARPGTRLSWLLCCSALLSPAAG
V-set and transmembrane domain containing
2B (hVSTM2B) signal peptide:
(SEQ ID NO: 4)
MEQRNRLGALGYLPPLLLHALLLFVADA
Protocadherin alpha-1 (hPCADHA1) signal peptide:
(SEQ ID NO: 5)
MVFSRRGGLGARDLLLWLLLLAAWEVGSG
FAM19A1 (TAFA1) signal peptide:
(SEQ ID NO: 6)
MAMVSAMSWVLYLWISACA
Interleukin-2 signal peptide:
(SEQ ID NO: 14)
MYRMQLLSCIALILALVTNS

Signal peptides may also be referred to herein as leader sequences or leader peptides.

The recombinant vector used for delivering the transgene should have a tropism for cells in the CNS, including but not limited to neurons and/or glial cells. Such vectors can include non-replicating recombinant adeno-associated virus vectors (“rAAV”), particularly those bearing an AAV9 or AAVrh10 capsid are preferred. AAV variant capsids can be used, including but not limited to those described by Wilson in U.S. Pat. No. 7,906,111 which is incorporated by reference herein in its entirety, with AAV/hu.31 and AAV/hu.32 being particularly preferred; as well as AAV variant capsids described by Chatterjee in U.S. Pat. Nos. 8,628,966, 8,927,514 and Smith et al., 2014, Mol Ther 22: 1625-1634, each of which is incorporated by reference herein in its entirety. However, other viral vectors may be used, including but not limited to lentiviral vectors, vaccinia viral vectors, or non-viral expression vectors referred to as “naked DNA” constructs (see Section 5.2).

Pharmaceutical compositions suitable for administration to the CSF comprise a suspension of the rHuGlyIDUA vector in a formulation buffer comprising a physiologically compatible aqueous buffer, a surfactant and optional excipients. In certain embodiments, the pharmaceutical compositions are suitable for intracisternal administration (injection into the cisterna magna). In certain embodiments, the pharmaceutical compositions are suitable for injection into the subarachnoid space via a C1-2 puncture. In certain embodiments, the pharmaceutical compositions are suitable for intrathecal administration. In certain embodiments, the pharmaceutical compositions are suitable for intracerebroventricular administration. In certain embodiments, the pharmaceutical compositions are suitable for administration via lumbar puncture.

Therapeutically effective doses of the recombinant vector should be administered to the CSF via intrathecal administration (i.e., injection into the subarachnoid space so that the recombinant vectors distribute through the CSF and transduce cells in the CNS). This can be accomplished in a number of ways—e.g., by intracranial (cisternal or ventricular) injection, or injection into the lumbar cistern. For example intracisternal (IC) injection (into the cisterna magna) can be performed by CT-guided suboccipital puncture; or injection into the subarachnoid space can be performed via a C1-2 puncture when feasible for the patient; or lumbar puncture (typically diagnostic procedures performed in order to collect a sample of CSF) can be used to access the CSF. Alternatively, intracerebroventricular (ICV) administration (a more invasive technique used for the introduction of antiinfective or anticancer drugs that do not penetrate the blood-brain barrier) can be used to instill the recombinant vectors directly into the ventricles of the brain. Alternatively, intranasal administration may be used to administer the recombinant vector to the CNS. Doses that maintain a CSF concentration of rHuGlyIDUA at a Cₘᵢₙ of at least 9.25 μg/mL or concentrations ranging from 9.25 to 277 μg/mL should be used.

CSF concentrations can be monitored by directly measuring the concentration of rHuGlyIDUA in the CSF fluid obtained from occipital or lumbar punctures, or estimated by extrapolation from concentrations of the rHuGlyIDUA detected in the patient's serum. In certain embodiments, 10 ng/mL to 100 ng/mL of rHuGlyIDUA in the serum is indicative of 1 to 30 mg of rHuGlyIDUA in the CSF. In certain embodiments, the recombinant vector is administered to the CSF at a dose that maintains 10 ng/mL to 100 ng/mL of rHuGlyIDUA in the serum.

By way of background, human IDUA is translated as a 653 amino acid polypeptide and is N-glycosylated at six potential sites (N110, N190, N336, N372, N415 and N451) depicted in FIG. 1. The signal sequence is removed and the polypeptide is processed into the mature form in lysosomes: a 75 kDa intracellular precursor is trimmed to 72 kDa in several hours, and eventually, over 4 to 5 days, is processed to a 66 kDa intracellular form. A secreted form of IDUA (76 kDa or 82 kDa depending on the assay used) is readily endocytosed by cells via the mannose-6-phosphate receptor and similarly processed to the smaller intracellular forms. (See, Myerowitz & Neufeld, 1981, J Biol Chem 256: 3044-3048; Clements et al., 1989, Biochem J. 259: 199-208; Taylor et al., 1991, Biochem J. 274: 263-268; and Zhao et al., 1997 J Biol Chem 272:22758-22765 each of which is incorporated by reference herein in its entirety).

The overall structure of hIDUA consists of three domains: residues 42-396 form a classic (β/α) triosephosphate isomerase (TIM) barrel domain; residues 27-42 and 397-545 form a β-sandwich domain with a short helix-loop-helix (482-508); and residues 546-642 form an Ig-like domain. The latter two domains are linked through a disulfide bridge between C⁵⁴¹ and C⁵⁷⁷. The β-sandwich and Ig-like domains are attached to the first, seventh, and eighth α-helices of the TIM barrel. A β-hairpin (β12-β13) is inserted between the eighth β-strand and the eighth α-helix of the TIM barrel, which includes N-glycosylated N³⁷² which is required for substrate binding and enzymatic activity. (See, FIG. 1 and crystal structure described in Maita et al., 2013, PNAS 110: 14628-14633, and Saito et al., 2014, Mol Genet Metab 111: 107-112 each of which is incorporated by reference herein in its entirety).

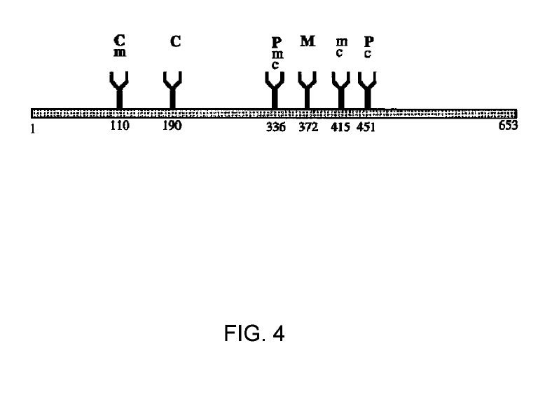

The invention is based, in part, on the following principles:
(i) Neuron and glial cells in the CNS are secretory cells that possess the cellular machinery for post-translational processing of secreted proteins—including glycosylation and tyrosine-O-sulfation robust processes in the CNS. See, e.g., Sleat et al., 2005, Proteomics 5: 1520-1532, and Sleat 1996, J Biol Chem 271: 19191-98 which describes the human brain mannose-6-phosphate (M6P) glycoproteome and notes that the brain contains more proteins with a much greater number of individual isoforms and mannose-6-phosphorylated proteins than found in other tissues; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by neuronal cells, each of which is incorporated by reference in its entirety for post-translational modifications made by human CNS cells.
(ii) hIDUA has six asparaginal (“N”) glycosylation sites identified in FIG. 1 (N¹¹⁰FT; N¹⁹⁰VS; N³³⁶TT; N³⁷²NT; N⁴¹⁵HT; N⁴⁵¹RS). N-glycosylation of N³⁷² is required for binding to substrate and enzymatic activity, and mannose-6-phosphorylation is required for cellular uptake of the secreted enzyme and cross-correction of MPS I cells. The N-linked glycosylation sites contain complex, high mannose and phosphorylated mannose carbohydrate moieties (FIG. 4), but only the secreted form is taken up by cells. (Myerowitz & Neufeld, 1981, supra). The gene therapy approach described herein should result in the continuous secretion of an IDUA glycoprotein of 76-82 kDa as measured by polyacrylamide gel electrophoresis (depending on the assay used) that is 2,6-sialylated and mannose-6-phosphorylated. The secreted glycosylated/phosphorylated IDUA should be taken up and correctly processed by untransduced neural and glial cells in the CNS.
(iii) The cellular and subcellular trafficking/uptake of lysosomal proteins is through M6P. It is possible to measure the M6P content of a secreted protein, as done in Daniele 2002 for the iduronate-2-sulfatase enzyme. In the presence of inhibitory M6P (e.g., 5 mM), the uptake of the enzyme precursor generated by non-neuronal or non-glial cells, such as the genetically engineered kidney cells of Daniele 2002, is predicted to decrease to levels close to that of the control cells, as was shown in Daniele 2002. While in the presence of inhibitory M6P, the uptake of enzyme precursor generated by brain cells, such as neuronal and glial cells, is predicted to remain at a high level, as was shown in Daniele 2002, where the uptake was four times higher than control cells and comparable to the level of enzyme activity (or uptake) of enzyme precursor generated by genetically engineered kidney cells without the presence of inhibitory M6P. This assay allows for a way to predict the M6P content in an enzyme precursor generated by brain cells, and, in particular, to compare the M6P content in enzyme precursors generated by different types of cells. The gene therapy approach described herein should result in the continuous secretion of hIDUA that may be taken up into neuronal and glial cells at a high level in the presence of inhibitory M6P in such an assay.
(iv) In addition to the N-linked glycosylation sites, hIDUA contains a tyrosine (“Y”) sulfation site (ADTPIY²⁹⁶NDEADPLVGWS) near the domain containing N³⁷² required for binding and activity. (See, e.g., Yang et al., 2015, Molecules 20:2138−2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). While not intending to be bound by any theory, sulfation of this site in hIDUA may be critical to activity since mutations within the tyrosine-sulfation region (e.g., W306L) are known to be associated with decreased enzymatic activity and disease. (See, Maita et al., 2013, PNAS 110:14628 at pp. 14632-14633).
(v) The glycosylation of hIDUA by human cells of the CNS will result in the addition of glycans that can improve stability, half-life and reduce unwanted aggregation of the transgene product. Significantly, the glycans that are added to HuGlyIDUA of the invention are highly processed complex-type biantennary N-glycans that include 2,6-sialic acid, incorporating Neu5Ac (“NANA”) but not its hydroxylated derivative, NeuGc (N-Glycolylneuraminic acid, i.e., “NGNA” or “Neu5Gc”). Such glycans are not present in laronidase which is made in CHO cells that do not have the 2,6-sialyltransferase required to make this post-translational modification, nor do CHO cells produce bisecting GlcNAc, although they do add Neu5Gc (NGNA) as sialic acid not typical (and potentially immunogenic) to humans instead of Neu5Ac (NANA). See, e.g., Dumont et al., 2016, Critical Rev in Biotech 36(6):1110-1122 (Early Online pp. 1-13 at p. 5); and Hague et al., 1998 Electrophor 19:2612-2630 (“[t]he CHO cell line is considered ‘phenotypically restricted,’ in terms of glycosylation, due to the lack of an α2,6-sialyl-transferase”). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuGlyIDUA of the invention should reduce immunogenicity of the transgene product and improve efficacy.
(vi) Tyrosine-sulfation of hIDUA—a robust post-translational process in human CNS cells—should result in improved processing and activity of transgene products. The significance of tyrosine-sulfation of lysosomal proteins has not been elucidated; but in other proteins it has been shown to increase avidity of protein-protein interactions (antibodies and receptors), and to promote proteolytic processing (peptide hormone). (See, Moore, 2003, J Biol. Chem. 278:24243-46; and Bundegaard et al., 1995, The EMBO J 14: 3073-79). The tyrosylprotein sulfotransferase (TPST1) responsible for tyrosine-sulfation (which may occur as a final step in IDUA processing) is apparently expressed at higher levels (based on mRNA) in the brain (gene expression data for TPST1 may be found, for example, at the EMBL-EBI Expression Atlas, accessible at http://www.ebi.ac.uk/gxa/home). Such post-translational modification, at best, is under-represented in laronidase—a CHO cell product. Unlike human CNS cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).
(vii) Immunogenicity of a transgene product could be induced by various factors, including the immune condition of the patient, the structure and characteristics of the infused protein drug, the administration route, and the duration of treatment. Process-related impurities, such as host cell protein (HCP), host cell DNA, and chemical residuals, and product-related impurities, such as protein degradants and structural characteristics, such as glycosylation, oxidation and aggregation (sub-visible particles), may also increase immunogenicity by serving as an adjuvant that enhances the immune response. The amounts of process-related and product-related impurities can be affected by the manufacturing process: cell culture, purification, formulation, storage and handling, which can affect commercially manufactured IDUA products. In gene therapy, proteins are produced in vivo, such that process-related impurities are not present and protein products are not likely to contain product-related impurities/degradants associated with proteins produced by recombinant technologies, such as protein aggregation and protein oxidation. Aggregation, for example, is associated with protein production and storage due to high protein concentration, surface interaction with manufacturing equipment and containers, and the purification process with certain buffer systems. But these conditions that promote aggregation are not present when a transgene is expressed in vivo. Oxidation, such as methionine, tryptophan and histidine oxidation, is also associated with protein production and storage, caused, for example, by stressed cell culture conditions, metal and air contact, and impurities in buffers and excipients. The proteins expressed in vivo may also oxidize in a stressed condition, but humans, like many organisms, are equipped with an antioxidation defense system, which not only reduces the oxidation stress, but can also repairs and/or reverses the oxidation. Thus, proteins produced in vivo are not likely to be in an oxidized form. Both aggregation and oxidation could affect the potency, PK (clearance) and can increase immunogenicity concerns. The gene therapy approach described herein should result in the continuous secretion of an hIDUA with a reduced immunogenicity compared to commercially manufactured products.

For the foregoing reasons, the production of HuGlyIDUA should result in a “biobetter” molecule for the treatment of MPS I accomplished via gene therapy—e.g., by administering a viral vector or other DNA expression construct encoding HuGlyIDUA to the CSF of a patient (human subject) diagnosed with an MPS I disease (including but not limited to Hurler, Hurler-Scheie, or Scheie) to create a permanent depot in the CNS that continuously supplies a fully human-glycosylated, mannose-6-phosphorylated, sulfated transgene product secreted by the transduced CNS cells. The HuGlyIDUA transgene product secreted from the depot into the CSF will be endocytosed by cells in the CNS, resulting in “cross-correction” of the enzymatic defect in the MPS I recipient cells.

It is not essential that every hIDUA molecule produced either in the gene therapy or protein therapy approach be fully glycosylated and sulfated. Rather, the population of glycoproteins produced should have sufficient glycosylation (including 2,6-sialylation and mannose-6-phophorylation) and sulfation to demonstrate efficacy. The goal of gene therapy treatment of the invention is to slow or arrest the progression of disease. Efficacy may be monitored by measuring cognitive function (e.g., prevention or decrease in neurocognitive decline); reductions in biomarkers of disease (such as GAG) in CSF and or serum; and/or increase in IDUA enzyme activity in CSF and/or serum. Signs of inflammation and other safety events may also be monitored.

As an alternative, or an additional treatment to gene therapy, the rHuGlyIDUA glycoprotein can be produced in human cell lines by recombinant DNA technology and the glycoprotein can be administered to patients diagnosed with MPS I systemically and/or into the CSF for ERT). Human cell lines that can be used for such recombinant glycoprotein production include but are not limited to HT-22, SK-N-MC, HCN-1A, HCN-2, NT2, SH-SY5y, hNSC11, ReNcell VM, human embryonic kidney 293 cells (HEK293), fibrosarcoma HT-1080, HKB-11, CAP, HuH-7, and retinal cell lines, PER.C6, or RPE to name a few (see, e.g., Dumont et al., 2016, Critical Rev in Biotech 36(6): 1110-1122 “Human cell lines for biopharmaceutical manufacturing: history, status, and future perspectives” which is incorporated by reference in its entirety for a review of the human cell lines that could be used for the recombinant production of the rHuGlyIDUA glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation.

While the delivery of rHuGlyIDUA should minimize immune reactions, the clearest potential source of toxicity related to CNS-directed gene therapy is generating immunity against the expressed hIDUA protein in human subjects who are genetically deficient for IDUA and, therefore, potentially not tolerant of the protein and/or the vector used to deliver the transgene.

Thus, in a preferred embodiment, it is advisable to co-treat the patient with immune suppression therapy—especially when treating patients with severe disease who have close to zero levels of IDUA (e.g., Hurler). Immune suppression therapies involving a regimen of tacrolimus or rapamycin (sirolimus), for example, in combination with mycophenolic acid and/or in combination with corticosteroids such as prednisolone and/or methylprednisolone, or other immune suppression regimens used in tissue transplantation procedures can be employed. Such immune suppression treatment may be administered during the course of gene therapy, and in certain embodiments, pre-treatment with immune suppression therapy may be preferred. Immune suppression therapy can be continued subsequent to the gene therapy treatment, based on the judgment of the treating physician, and may thereafter be withdrawn when immune tolerance is induced; e.g., after 180 days.

In one embodiment, immune suppression comprises administration of a corticosteroid such as prednisolone and/or methylprednisolone and a regiment of tacrolimus and/or sirolimus, optionally administered with MMF. For example, one shot of a corticosteroid such as methylprednisolone is injected, followed by administration of an oral corticosteroid which is gradually tapered off over the course of 12 weeks and then discontinued. Concurrently, tacrolimus and sirolimus may be administered orally in combination at a low dose (e.g., maintaining 4 to 8 ng/mL serum concentration), or alone at the label dose, over 24 to 48 weeks. Tacrolimus or sirolimus may also be administered at the label dose in combination with MMF. Thus, the patient receives an initial injection of a steroid, which is available immediately, which steroid is then maintained through oral administration and tapered off by 12 weeks. Further immune suppression through 48 weeks is maintained by tacrolimus and/or sirolimus, optionally in combination with MMF.

Combinations of delivery of the HuGlyIDUA to the CSF accompanied by delivery of other available treatments are encompassed by the methods of the invention. The additional treatments may be administered before, concurrently or subsequent to the gene therapy treatment. Available treatments for MPS I that could be combined with the gene therapy of the invention include but are not limited to enzyme replacement therapy using laronidase administered systemically or to the CSF; and/or HSCT therapy.

In certain embodiments, described herein is a method for treating a human subject diagnosed with MPS I, comprising delivering to the cerebrospinal fluid of the brain of said human subject a therapeutically effective amount of recombinant human IDUA produced by human neuronal cells. In certain embodiments, described herein is a method for treating a human subject diagnosed with MPS I, comprising delivering to the cerebrospinal fluid of the brain of said human subject a therapeutically effective amount of recombinant human IDUA produced by human glial cells.

In certain embodiments, provided herein are methods of treating a human subject diagnosed with mucopolysaccharidosis I (MPS I), comprising: delivering to the cerebrospinal fluid of the brain of said human subject, a therapeutically effective amount of a α2,6-sialylated human α-L-iduronidase (IDUA); and administering an immune suppression therapy to said subject before or concurrently with the human IDUA treatment and continuing immune suppression therapy thereafter.

In certain embodiments, provided herein are methods of treating a human subject diagnosed with MPS I, comprising: delivering to the cerebrospinal fluid of the brain of said human subject, a therapeutically effective amount of a glycosylated human IDUA that does not contain detectable NeuGc; and administering an immune suppression therapy to said subject before or concurrently with the human IDUA treatment and continuing immune suppression therapy thereafter.

In certain embodiments, provided herein are methods of treating a human subject diagnosed with MPS I, comprising: delivering to the cerebrospinal fluid of the brain of said human subject, a therapeutically effective amount of a glycosylated human IDUA that does not contain detectable NeuGc and/or α-Gal antigen; and administering an immune suppression therapy to said subject before or concurrently with the human IDUA treatment and continuing immune suppression therapy thereafter.

In certain embodiments, provided herein are methods of treating a human subject diagnosed with MPS I, comprising: delivering to the cerebrospinal fluid of the brain of said human subject, a therapeutically effective amount of human IDUA that contains tyrosine-sulfation; and administering an immune suppression therapy to said subject before or concurrently with the human IDUA treatment and continuing immune suppression therapy thereafter.

In certain embodiments, provided herein are methods of treating a human subject diagnosed with MPS I, comprising: administering to the brain of said human subject an expression vector encoding human IDUA, wherein said IDUA is α2,6-sialylated upon expression from said expression vector in a human, immortalized neuronal cell; and

administering an immune suppression therapy to said subject before or concurrently with the administration of the expression vector and continuing immune suppression therapy thereafter.

In certain embodiments, provided herein are methods of treating a human subject diagnosed with MPS I, comprising: administering to the brain of said human subject an expression vector encoding human IDUA, wherein said IDUA is glycosylated but does not contain detectable NeuGc upon expression from said expression vector in a human, immortalized neuronal cell; and administering an immune suppression therapy to said subject before or concurrently with the administration of the expression vector and continuing immune suppression therapy thereafter.

In certain embodiments, provided herein are methods of treating a human subject diagnosed with MPS I, comprising: step a) administering to the brain of said human subject an expression vector encoding human IDUA, wherein said human IDUA is glycosylated but does not contain detectable NeuGc and/or α-Gal antigen upon expression from said expression vector in a human, or in an immortalized neuronal cell; and step b) administering an immune suppression therapy to said subject before and/or concurrently with and/or after the administration of the expression vector and continuing immune suppression therapy thereafter.

In certain embodiments, provided herein are methods of treating a human subject diagnosed with MPS I, comprising: administering to the brain of said human subject an expression vector encoding human IDUA, wherein said IDUA is tyrosine-sulfated upon expression from said expression vector in a human, immortalized neuronal cell; and administering an immune suppression therapy to said subject before or concurrently with the administration of the expression vector and continuing immune suppression therapy thereafter.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases said IDUA containing a α2,6-sialylated glycan; and administering an immune suppression therapy to said subject before or concurrently with the administration of the expression vector and continuing immune suppression therapy thereafter.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc; and administering an immune suppression therapy to said subject before or concurrently with the administration of the expression vector and continuing immune suppression therapy thereafter.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc and/or α-Gal antigen; and administering an immune suppression therapy to said subject before or concurrently with the administration of the expression vector and continuing immune suppression therapy thereafter.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases said IDUA containing a tyrosine-sulfation; and administering an immune suppression therapy to said subject before or concurrently with the administration of the expression vector and continuing immune suppression therapy thereafter.

In certain embodiments, production of said IDUA containing a α2,6-sialylated glycan is confirmed by transducing a human neuronal cell line with said recombinant nucleotide expression vector in cell culture. In certain embodiments, production of said glycosylated IDUA that does not contain detectable NeuGc is confirmed by transducing a human neuronal cell line with said recombinant nucleotide expression vector in cell culture. In certain embodiments, production of said glycosylated IDUA that does not contain detectable NeuGc and/or α-Gal antigen is confirmed by transducing a human neuronal cell line with said recombinant nucleotide expression vector in cell culture. In certain embodiments, production of said IDUA containing a tyrosine-sulfation is confirmed by transducing a human neuronal cell line with said recombinant nucleotide expression vector in cell culture. In specific embodiments, the IDUA transgene encodes a signal peptide. In certain embodiments, the human neuronal cell line is HT-22, SK-N-MC, HCN-1A, HCN-2, NT2, SH-SY5y, hNSC11, or ReNcell VM.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases said IDUA containing a α2,6-sialylated glycan; and administering an immune suppression therapy to said subject before or concurrently with the administration of the expression vector and continuing immune suppression therapy thereafter; wherein said recombinant vector, when used to transduce human neuronal cells in culture results in production of said IDUA containing said 2,6-sialylated glycan in said cell culture. In certain embodiments, the human neuronal cells are HT-22, SK-N-MC, HCN-1A, HCN-2, NT2, SH-SY5y, hNSC11, or ReNcell VM cells.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc; and administering an immune suppression therapy to said subject before or concurrently with the administration of the expression vector and continuing immune suppression therapy thereafter; wherein said recombinant vector, when used to transduce human neuronal cells in culture results in production of said IDUA that is glycosylated but does not contain detectable NeuGc in said cell culture. In certain embodiments, the human neuronal cells are HT-22, SK-N-MC, HCN-1A, HCN-2, NT2, SH-SY5y, hNSC11, or ReNcell VM cells.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc and/or α-Gal antigen; and administering an immune suppression therapy to said subject before or concurrently with the administration of the expression vector and continuing immune suppression therapy thereafter; wherein said recombinant vector, when used to transduce human neuronal cells in culture results in production of said IDUA that is glycosylated but does not contain detectable NeuGc and/or α-Gal antigen in said cell culture. In certain embodiments, the human neuronal cells are HT-22, SK-N-MC, HCN-1A, HCN-2, NT2, SH-SY5y, hNSC11, or ReNcell VM cells.

In certain embodiments, provided herein are methods of treating a human subject diagnosed with MPS I, comprising: administering to the cerebrospinal fluid of the brain of said human subject, a therapeutically effective amount of a recombinant nucleotide expression vector encoding human IDUA, so that a depot is formed that releases said IDUA that contains a tyrosine-sulfation; and administering an immune suppression therapy to said subject before or concurrently with the administration of the expression vector and continuing immune suppression therapy thereafter; wherein said recombinant vector, when used to transduce human neuronal cells in culture results in production of said IDUA that is tyrosine-sulfated in said cell culture. In certain embodiments, the human neuronal cells are HT-22, SK-N-MC, HCN-1A, HCN-2, NT2, SH-SY5y, hNSC11, or ReNcell VM cells.

In certain embodiments, the human IDUA comprises the amino acid sequence of SEQ ID NO. 1. In certain embodiments, the immune suppression therapy comprises administering a combination of (a) tacrolimus and mycophenolic acid, or (b) rapamycin and mycophenolic acid to said subject before or concurrently with the human IDUA treatment and continuing thereafter. In certain embodiments, the immune suppression therapy is withdrawn after 180 days.

In preferred embodiments, the glycosylated IDUA does not contain detectable NeuGc and/or α-Gal. The phrase “detectable NeuGc and/or α-Gal” used herein means NeuGc and/or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377: 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring alpha-Gal epitope expression on cells by a monoclonal anti-Gal antibody.” Transplantation. 65(8): 1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5): 699-710. See also the references cited in Platts-Mills et al., 2015, “Anaphylaxis to the Carbohydrate Side-Chain Alpha-gal” Immunol Allergy Clin North Am. 35(2): 247-260.

5.1 N-Glycosylation and Tyrosine Sulfation

5.1.1. N-Glycosylation

Neuron and glial cells in the CNS are secretory cells that possess the cellular machinery for post-translational processing of secreted proteins—including glycosylation and tyrosine-O-sulfation. hIDUA has six asparaginal (“N”) glycosylation sites identified in FIG. 1 (N¹¹⁰FT; N¹⁹⁰VS; N³³⁶TT; N³⁷²NT; N⁴¹⁵HT; N⁴⁵¹RS). N-glycosylation of N³⁷² is required for binding to substrate and enzymatic activity, and mannose-6-phosphorylation is required for cellular uptake of the secreted enzyme and cross-correction of MPS I cells. The N-linked glycosylation sites contain complex, high mannose and phosphorylated mannose carbohydrate moieties (FIG. 4), but only the secreted form is taken up by cells. The gene therapy approach described herein should result in the continuous secretion of an IDUA glycoprotein that is 2,6-sialylated and mannose-6-phosphorylated. The secreted glycosylated/phosphorylated IDUA should be taken up and correctly processed by untransduced neural and glial cells in the CNS.

The glycosylation of hIDUA by human cells of the CNS will result in the addition of glycans that can improve stability, half-life and reduce unwanted aggregation of the transgene product. Significantly, the glycans that are added to HuGlyIDUA of the invention are highly processed complex-type biantennary N-glycans that include 2,6-sialic acid, incorporating Neu5Ac (“NANA”) but not its hydroxylated derivative, NeuGc (N-Glycolylneuraminic acid, i.e., “NGNA” or “Neu5Gc”). Such glycans are not present in laronidase which is made in CHO cells that do not have the 2,6-sialyltransferase required to make this post-translational modification, nor do CHO cells produce bisecting GlcNAc, although they do add Neu5Gc (NGNA) as sialic acid not typical (and potentially immunogenic) to humans instead of Neu5Ac (NANA). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuGlyIDUA of the invention should reduce immunogenicity of the transgene product and improve efficacy.

It is not essential that every molecule produced either in the gene therapy or protein therapy approach be fully glycosylated and sulfated. Rather, the population of glycoproteins produced should have sufficient glycosylation and sulfation to demonstrate efficacy.

In a specific embodiment, HuGlyIDUA used in accordance with the methods described herein, when expressed in a neuron or glial cell, in vivo or in vitro, could be glycosylated at 100% of its N-glycosylation sites. However, one of skill in the art will appreciate that not every N-glycosylation site of HuGlyIDUA need be N-glycosylated in order for benefits of glycosylation to be attained. Rather, benefits of glycosylation can be realized when only a percentage of N-glycosylation sites are glycosylated, and/or when only a percentage of expressed IDUA molecules are glycosylated. Accordingly, in certain embodiments, HuGlyIDUA used in accordance with the methods described herein, when expressed in a neuron or glial cell, in vivo or in vitro, is glycosylated at 10%-20%, 20%-30%, 30%-40%, 40%-50%, 50%-60%, 60%-70%, 70%-80%, 80%-90%, or 90%-100% of its available N-glycosylation sites. In certain embodiments, when expressed in a neuron or glial cell, in vivo or in vitro, 10%-20%, 20%-30%, 30%-40%, 40%-50%, 50%-60%, 60%-70%, 70%-80%, 80%-90%, or 90%-100% of HuGlyIDUA molecules used in accordance with the methods described herein are glycosylated at least one of their available N-glycosylation sites.

In a specific embodiment, at least 10%, 20% 30%, 40%, 50%, 60%, 70%, 75%, 80%, 85%, 90%, 95%, or 99% of the N-glycosylation sites present in HuGlyIDUA used in accordance with the methods described herein are glycosylated at an Asn residue (or other relevant residue) present in an N-glycosylation site, when the HuGlyIDUA is expressed in a neuron or glial cell, in vivo or in vitro. That is, at least 50%, 60%, 70%, 75%, 80%, 85%, 90%, 95%, or 99% of the N-glycosylation sites of the resultant HuGlyIDUA are glycosylated.

In another specific embodiment, at least 10%, 20% 30%, 40%, 50%, 60%, 70%, 75%, 80%, 85%, 90%, 95%, or 99% of the N-glycosylation sites present in a HuGlyIDUA molecule used in accordance with the methods described herein are glycosylated with an identical attached glycan linked to the Asn residue (or other relevant residue) present in an N-glycosylation site, when the HuGlyIDUA is expressed n a neuron or glial cell, in vivo or in vitro. That is, at least 50%, 60%, 70%, 75%, 80%, 85%, 90%, 95%, or 99% of the N-glycosylation sites of the resultant HuGlyIDUA have an identical attached glycan.

Importantly, when the IDUA proteins used in accordance with the methods described herein are expressed in neuron or glial cells, the need for in vitro production in prokaryotic host cells (e.g., E. coli) or eukaryotic host cells (e.g., CHO cells) is circumvented. Instead, as a result of the methods described herein (e.g., use of neuron or glial cells to express IDUA), N-glycosylation sites of the IDUA proteins are advantageously decorated with glycans relevant to and beneficial to treatment of humans. Such an advantage is unattainable when CHO cells or E. coli are utilized in protein production, because e.g., CHO cells (1) do not express 2,6 sialyltransferase and thus cannot add 2,6 sialic acid during N-glycosylation and (2) can add Neu5Gc as sialic acid instead of Neu5Ac; and because E. coli does not naturally contain components needed for N-glycosylation. Accordingly, in one embodiment, an IDUA protein expressed in a neuron or glial cell to give rise to a HuGlyIDUA used in the methods of treatment described herein is glycosylated in the manner in which a protein is N-glycosylated in human neuron or glial cells, but is not glycosylated in the manner in which proteins are glycosylated in CHO cells. In another embodiment, an IDUA protein expressed in a neuron or glial cell to give rise to a HuGlyIDUA used in the methods of treatment described herein is glycosylated in the manner in which a protein is N-glycosylated in a neuron or glial cells, wherein such glycosylation is not naturally possible using a prokaryotic host cell, e.g., using E. coli.

Assays for determining the glycosylation pattern of proteins are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low molecular weight can be analyzed by MALDI-MS/MS and the result used to confirm the glycan sequence. Each peak corresponds to a polymer consisting of a certain number of repeat units and fragments thereof. The chromatogram thus allows measurement of the polymer length distribution. The elution time is an indication for polymer length, while fluorescence intensity correlates with molar abundance for the respective polymer.

Homogeneity of the glycan patterns associated with proteins, as it relates to both glycan length and numbers glycans present across glycosylation sites, can be assessed using methods known in the art, e.g., methods that measure glycan length and hydrodynamic radius. Size exclusion-HPLC allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

N-glycosylation confers numerous benefits on the HuGlyIDUA used in the methods described herein. Such benefits are unattainable by production of proteins in E. coli, because E. coli does not naturally possess components needed for N-glycosylation. Further, some benefits are unattainable through protein production in, e.g., CHO cells, because CHO cells lack components needed for addition of certain glycans (e.g., 2,6 sialic acid) and because CHO cells can add glycans, e.g., Neu5Gc not typical to humans, and the α-Gal antigen which is immunogenic in most individuals and at high concentrations can trigger anaphylaxis. Thus, the expression of IDUA in human neuron or glial cells results in the production of HuGlylDUA comprising beneficial glycans that otherwise would not be associated with the protein if produced in CHO cells or in E. coli.

5.1.2. Tyrosine Sulfation

In addition to the N-linked glycosylation sites, hIDUA contains a tyrosine (“Y”) sulfation site (ADTPIY²⁹⁶NDEADPLVGWS) near the domain containing N³⁷² required for binding and activity. (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). While not intending to be bound by any theory, sulfation of this site in hIDUA may be critical to activity since mutations within the tyrosine-sulfation region (e.g., W306L) are known to be associated with decreased enzymatic activity and disease. (See, Maita et al., 2013, PNAS 110:14628 at pp. 14632-14633).

Importantly, tyrosine-sulfated proteins cannot be produced in E. coli, which naturally does not possess the enzymes required for tyrosine-sulfation. Further, CHO cells are deficient for tyrosine sulfation-they are not secretory cells and have a limited capacity for post-translational tyrosine-sulfation. See, e.g., Mikkelsen & Ezban, 1991, Biochemistry 30: 1533-1537. Advantageously, the methods provided herein call for expression of IDUA, e.g., HuGlyIDUA, in neurons or glial cells, which are secretory and do have capacity for tyrosine sulfation. Assays for detection tyrosine sulfation are known in the art. See, e.g., Yang et al., 2015, Molecules 20:2138-2164.

Tyrosine-sulfation of hIDUA—a robust post-translational process in human CNS cells—should result in improved processing and activity of transgene products. The significance of tyrosine-sulfation of lysosomal proteins has not been elucidated; but in other proteins it has been shown to increase avidity of protein-protein interactions (antibodies and receptors), and to promote proteolytic processing (peptide hormone). (See, Moore, 2003, J Biol. Chem. 278:24243-46; and Bundegaard et al., 1995, The EMBO J 14: 3073-79). The tyrosylprotein sulfotransferase (TPST1) responsible for tyrosine-sulfation (which may occur as a final step in IDUA processing) is apparently expressed at higher levels (based on mRNA) in the brain (gene expression data for TPST1 may be found, for example, at the EMBL-EBI Expression Atlas, accessible at http://www.ebi.ac.uk/gxa/home). Such post-translational modification, at best, is under-represented in laronidase—a CHO cell product.

5.2 Constructs and Formulations

For use in the methods provided herein are viral vectors or other DNA expression constructs encoding α-L-iduronidase (IDUA), e.g., human IDUA (hIDUA). The viral vectors and other DNA expression constructs provided herein include any suitable method for delivery of a transgene to the cerebrospinal fluid (CSF). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to neuronal cells.

In some aspects, the disclosure provides for a nucleic acid for use, wherein the nucleic acid encodes an IDUA, e.g., hIDUA, operatively linked to a promoter selected from the group consisting of: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., IDUA), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

5.2.1. mRNA

In certain embodiments, the vectors provided herein are modified mRNA encoding for the gene of interest (e.g., the transgene, for example, IDUA). The synthesis of modified and unmodified mRNA for delivery of a transgene to the CSF is taught, for example, in Hocquemiller et al., 2016, Human Gene Therapy 27(7):478-496, which is incorporated by reference herein in its entirety. In certain embodiments, provided herein is a modified mRNA encoding for IDUA, e.g., hIDUA. 5.2.2. Viral Vectors

Viral vectors include adenovirus, adeno-associated virus (AAV, e.g., AAV9), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

In certain embodiments, the viral vectors provided herein are AAV based viral vectors. In preferred embodiments, the viral vectors provided herein are AAV9 or AAVrh10 based viral vectors. In certain embodiments, the AAV9 or AAVrh10 based viral vectors provided herein retain tropism for CNS cells. Multiple AAV serotypes have been identified. In certain embodiments, AAV-based vectors provided herein comprise components from one or more serotypes of AAV. In certain embodiments, AAV based vectors provided herein comprise components from one or more of AAV1, AAV2, AAV3, AAV4, AAV5, AAV6, AAV7, AAV8, AAV9, AAVrh10, or AAV11. In preferred embodiments, AAV based vectors provided herein comprise components from one or more of AAV8, AAV9, AAV10, or AAV11 serotypes. AAV9-based viral vectors are used in the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV9 or AAVrh10)-based viral vectors encoding a transgene (e.g., IDUA). In specific embodiments, provided herein are AAV9-based viral vectors encoding IDUA. In more specific embodiments, provided herein are AAV9-based viral vectors encoding hIDUA.

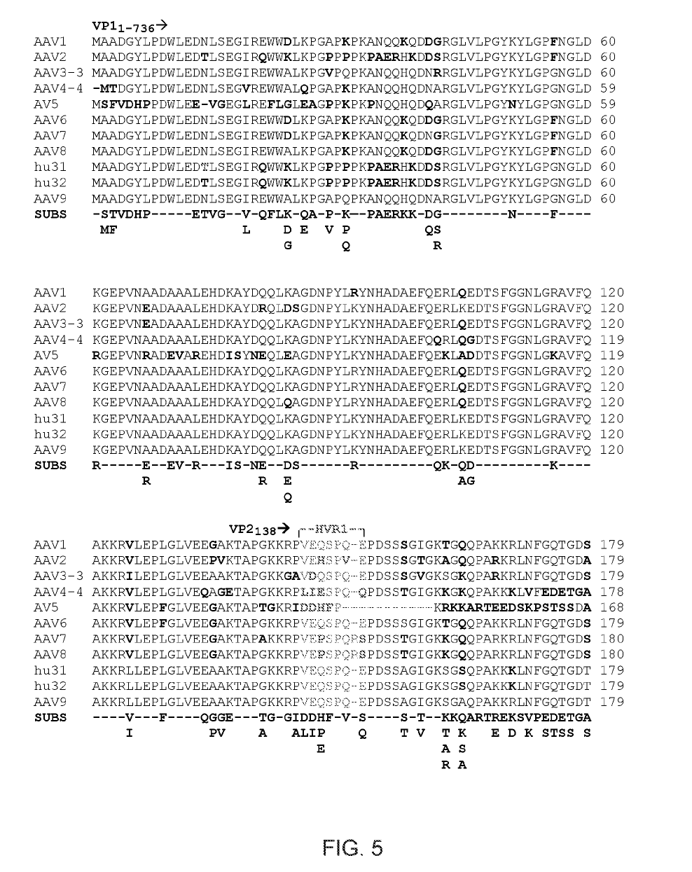

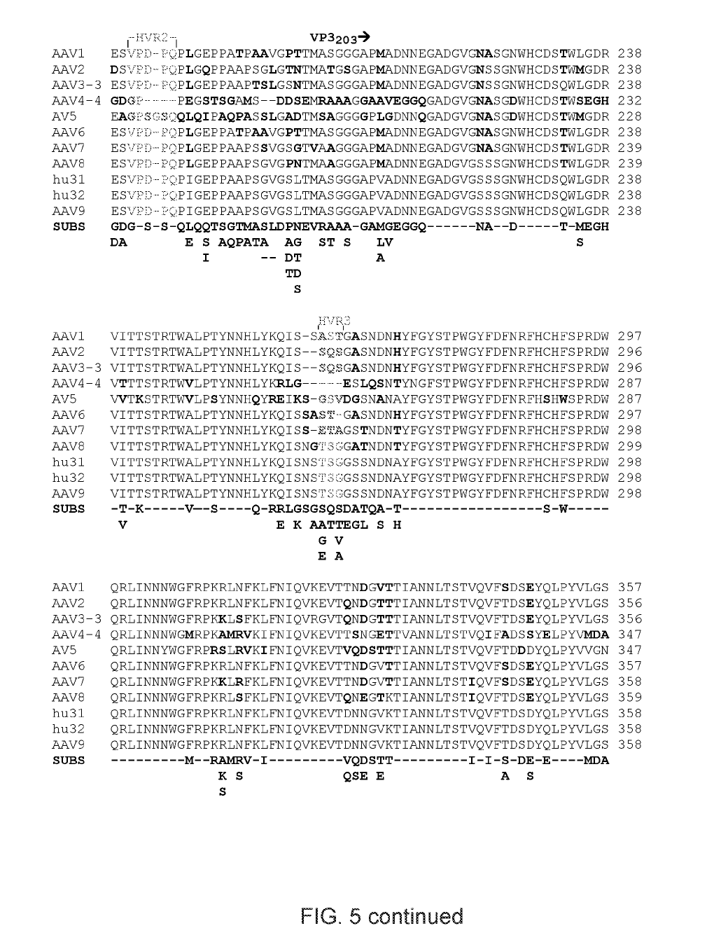

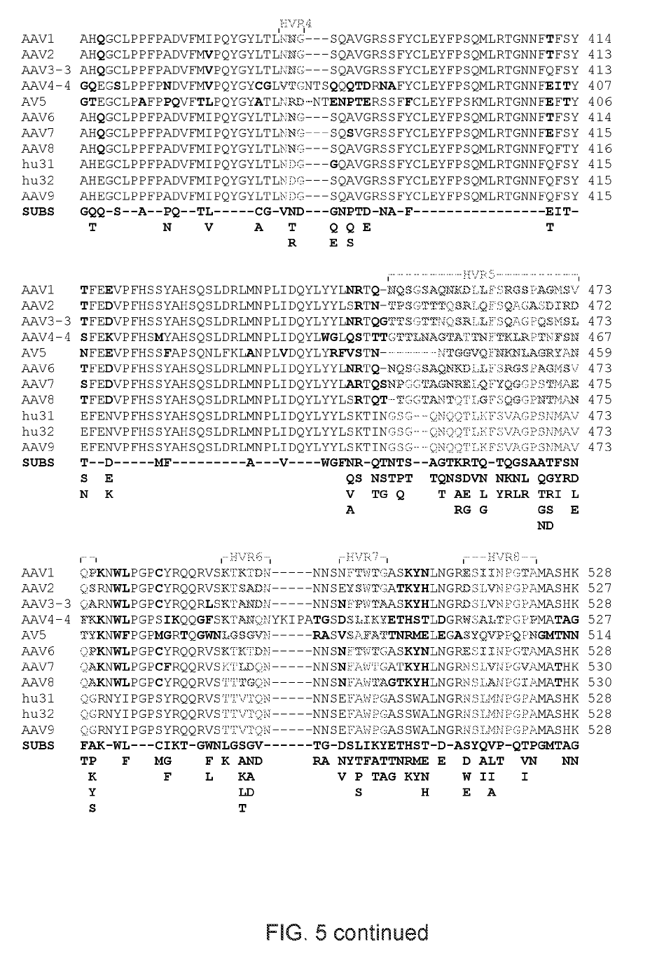

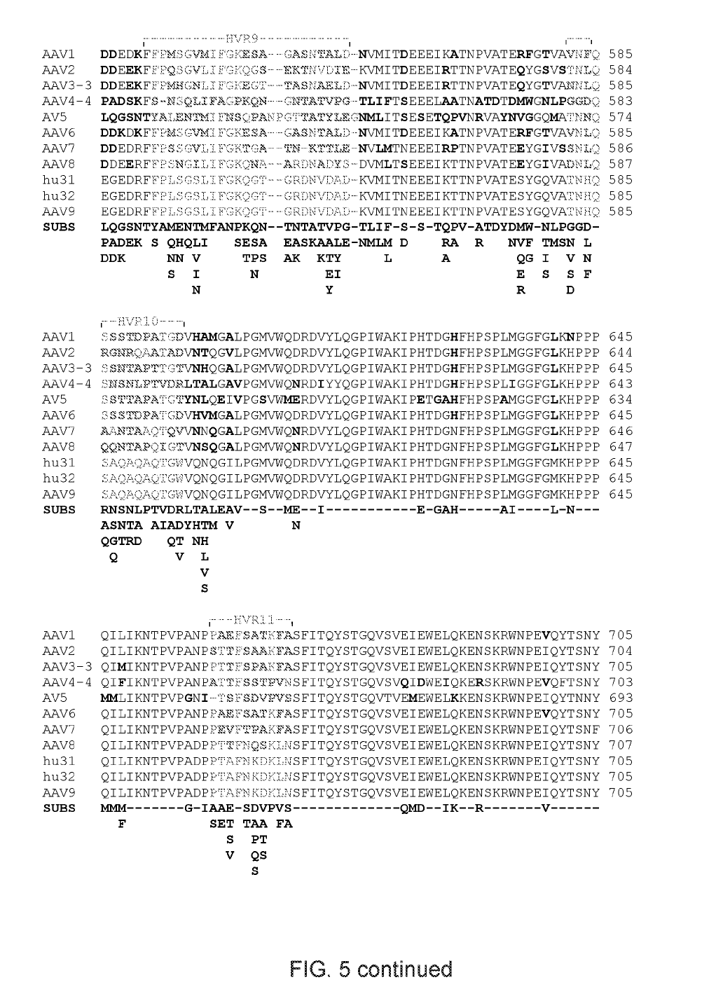

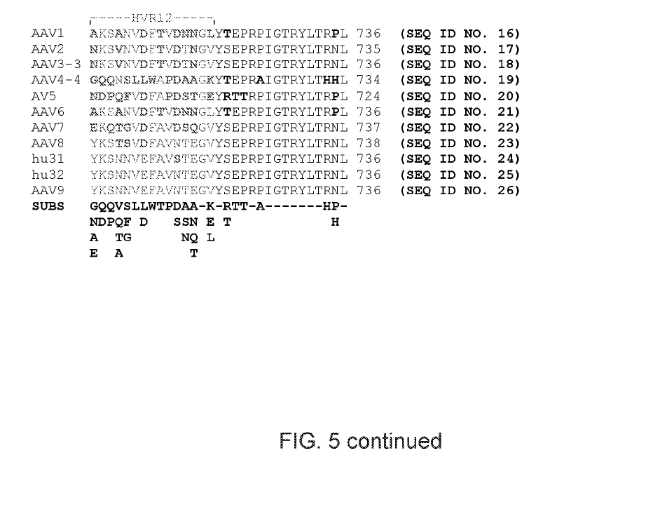

Provided in particular embodiments are AAV9 vectors comprising an artificial genome comprising (i) an expression cassette containing the transgene under the control of regulatory elements and flanked by ITRs; and (ii) a viral capsid that has the amino acid sequence of the AAV9 capsid protein or is at least 95%, 96%, 97%, 98%, 99% or 99.9% identical to the amino acid sequence of the AAV9 capsid protein (SEQ ID NO: 26) while retaining the biological function of the AAV9 capsid. In certain embodiments, the encoded AAV9 capsid has the sequence of SEQ ID NO: 26 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid. FIG. 5 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV9 vector comprises an AAV9 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 5 that are not present at that position in the native AAV9 sequence.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the IDUA. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the artificial genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the IDUA. Four plasmids are used to make the construct: Gag/pol sequence containing plasmid, Rev sequence containing plasmids, Envelope protein containing plasmid (i.e. VSV-G), and Cis plasmid with the packaging elements and the IDUA gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an IDUA (e.g., hIDUA) such that, upon transduction of cells in the CNS, or a relevant cell (e.g., a neuronal cell in vivo or in vitro), a glycosylated variant of IDUA is expressed by the transduced cell. In a specific embodiment, a vector for use in the methods described herein is one that encodes an IDUA (e.g., hIDUA) such that, upon transduction of a cell in the CNS, or a relevant cell (e.g., a neuronal cell in vivo or in vitro), a sulfated variant of IDUA is expressed by the cell.

5.2.3. Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In alternate embodiments, the promoter is an inducible promoter. The native IDUA gene, like most housekeeping genes, primarily uses a GC-rich promoter. In a preferred embodiment, strong constitutive promoters that provide for sustained expression of hIDUA are used. Such promoters include “CAG” synthetic promoters that contain: “C”—the cytomegalovirus (CMV) early enhancer element; “A”—the promoter as well as the first exon and intron of the chicken beta-actin gene; and “G”—the splice acceptor of the rabbit beta-globin gene (see, Miyazaki et al., 1989, Gene 79: 269-277; and Niwa et al., Gene 108: 193-199).

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a neuronal cell-specific promoter).

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

5.2.4. Signal Peptides

In certain embodiments, the vectors provided herein comprise components that modulate protein delivery. In certain embodiments, the viral vectors provided herein comprise one or more signal peptides. In certain embodiments, the signal peptides allow for the transgene product (e.g., IDUA) to achieve the proper packaging (e.g. glycosylation) in the cell. In certain embodiments, the signal peptides allow for the transgene product (e.g., IDUA) to achieve the proper localization in the cell. In certain embodiments, the signal peptides allow for the transgene product (e.g., IDUA) to achieve secretion from the cell. Examples of signal peptides to be used in connection with the vectors and transgenes provided herein may be found in Table 1. Signal peptides may also be referred to herein as leader sequences or leader peptides.

TABLE 1
Signal peptides for use
with the vectors provided herein.

| SEQ | | |
|---|---|---|
| ID | | |
| NO. | Signal Peptide | Sequence |
| 2 | Oligodendrocyte- | MEYQILKMSLCLFILLFLTPGILC |
| | myelin | |
| | glycoprotein | |
| | (hOMG) signal | |
| | peptide | |
| 3 | Cellular | MSVRRGRRPARPGTRLSWLLCCSA |
| | repressor of | LLSPAAG |
| | E1A-stimulated | |
| | genes 2 (hCREG2) | |
| | signal peptide | |
| 4 | V-set and | MEQRNRLGALGYLPPLLLHALLLF |
| | transmembrane | VADA |
| | domain | |
| | containing 2B | |
| | (hVSTM2B) | |
| | signal peptide | |
| 5 | Protocadherin | MVFSRRGGLGARDLLLWLLLLAAW |
| | alpha-1 | EVGSG |
| | (hPCADHA1) | |
| | signal peptide | |
| 6 | FAM19A1 (TAFA1) | MAMVSAMSWVLYLWISACA |
| | signal peptide | |
| 7 | VEGF-A signal | MNFLLSWVHWSLALLLYLHHAKWS |
| | peptide | QA |
| 8 | Fibulin-1 | MERAAPSRRVPLPLLLLGGLALLA |
| | signal peptide | AGVDA |
| 9 | Vitronectin | MAPLRPLLILALLAWVALA |
| | signal peptide | |
| 10 | Complement | MRLLAKIICLMLWAICVA |
| | Factor H | |
| | signal peptide | |
| 11 | Opticin signal | MRLLAFLSLLALVLQETGT |
| | peptide | |
| 12 | Albumin signal | MKWVTFISLLFLFSSAYS |
| | peptide | |
| 13 | Chymotrypsinogen | MAFLWLLSCWALLGTTFG |
| | signal peptide | |
| 14 | Interleukin-2 | MYRMQLLSCIALILALVTNS |
| | signal peptide | |
| 15 | Trypsinogen-2 | MNLLLILTFVAAAVA |
| | signal peptide | |

5.2.5. Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half life of the transgene. In certain embodiments, the UTRs are optimized for the stability of the mRNA of the transgene. In certain embodiments, the UTRs are optimized for the secondary structure of the mRNA of the transgene.

5.2.6. Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV9 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

5.2.7. Transgenes

In certain embodiments, the vectors provided herein encode an IDUA transgene. In specific embodiments, the IDUA is controlled by appropriate expression control elements for expression in neuronal cells: In certain embodiments, the IDUA (e.g., hIDUA) transgene comprises the amino acid sequence of SEQ ID NO: 1. In certain embodiments, the IDUA (e.g., hIDUA) transgene comprises an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1.

The HuGlyIDUA encoded by the transgene can include, but is not limited to human IDUA (hIDUA) having the amino acid sequence of SEQ ID NO. 1 (as shown in FIG. 1), and derivatives of hIDUA having amino acid substitutions, deletions, or additions, e.g., including but not limited to amino acid substitutions selected from corresponding non-conserved residues in orthologs of IDUA shown in FIG. 2, with the proviso that such mutations do not include any that have been identified in severe, severe-intermediate, intermediate, or attenuated MPS I phenotypes shown in FIG. 3 (from, Saito et al., 2014, Mol Genet Metab 111:107-112, Table 1 listing 57 MPS I mutations, which is incorporated by reference herein in its entirety); or reported by Venturi et al., 2002, Human Mutation #522 Online (“Venturi 2002”), or Bertola et al., 2011 Human Mutation 32:E2189-E2210 (“Bertola 2011”), each of which is incorporated by reference herein in its entirety.

For example, amino acid substitutions at a particular position of hIDUA can be selected from among corresponding non-conserved amino acid residues found at that position in the IDUA orthologs depicted in FIG. 2 (showing alignment of orthologs as reported Maita et al., 2013, PNAS 110:14628, FIG. S8 which is incorporated by reference herein in its entirety), with the proviso that such substitutions do not include any of the deleterious mutations shown in FIG. 3 or reported in Venturi 2002 or Bertola 2011 supra. The resulting transgene product can be tested using conventional assays in vitro, in cell culture or test animals to ensure that the mutation does not disrupt IDUA function. Preferred amino acid substitutions, deletions or additions selected should be those that maintain or increase enzyme activity, stability or half-life of IDUA, as tested by conventional assays in vitro, in cell culture or animal models for MPS I. For example, the enzyme activity of the transgene product can be assessed using a conventional enzyme assay with 4-methylumbelliferyl α-L-iduronide as the substrate (see, e.g., Hopwood et al., 1979, Clin Chim Acta 92: 257-265; Clements et al., 1985, Eur J Biochem 152: 21-28; and Kakkis et al., 1994, Prot Exp Purif 5: 225-232 for exemplary IDUA enzyme assays that can be used, each of which is incorporated by reference herein in its entirety). The ability of the transgene product to correct MPS I phenotype can be assessed in cell culture; e.g., by transducing MPS I cells in culture with a viral vector or other DNA expression construct encoding hIDUA or a derivative; by adding the rHuGlyIDUA or a derivative to MPS I cells in culture; or by co-culturing MPS I cells with human host cells engineered to express and secrete rHuGlyIDUA or a derivative, and determining correction of the defect in the MPS I cultured cells, e.g., by detecting IDUA enzyme activity and/or reduction in GAG storage in the MPS I cells in culture (see e.g., Myerowitz & Neufeld, 1981, J Biol Chem 256: 3044-3048; and Anson et al. 1992, Hum Gene Ther 3: 371-379, each of which is incorporated by reference herein in its entirety).

5.2.8. Constructs

In certain embodiments, the viral vectors provided herein comprise the following elements in the following order: a) a first ITR sequence, b) a first linker sequence, c) a promoter sequence, d) a second linker sequence, e) an intron sequence, f) a third linker sequence, g) a sequence encoding the transgene (e.g., IDUA), h) a fourth linker sequence, i) a poly A sequence, j) a fifth linker sequence, and k) a second ITR sequence.

In certain embodiments, the viral vectors provided herein comprise the following elements in the following order: a) a promoter sequence, and b) a sequence encoding the transgene (e.g., IDUA). In certain embodiments, the viral vectors provided herein comprise the following elements in the following order: a) a promoter sequence, and b) a sequence encoding the transgene (e.g., IDUA), wherein the transgene comprises a signal peptide.

In certain embodiments, the viral vectors provided herein comprise the following elements in the following order: a) a first ITR sequence, b) a first linker sequence, c) a promoter sequence, d) a second linker sequence, e) an intron sequence, f) a third linker sequence, g) a first UTR sequence, h) a sequence encoding the transgene (e.g., IDUA), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a promoter sequence, d) a second linker sequence, e) an intron sequence, f) a third linker sequence, g) a first UTR sequence, h) a sequence encoding the transgene (e.g., IDUA), i) a second UTR sequence, j) a fourth linker sequence, k) a poly A sequence, 1) a fifth linker sequence, and m) a second ITR sequence, wherein the transgene comprises a signal peptide, and wherein the transgene encodes hIDUA.

5.2.9. Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

In vitro assays, e.g., cell culture assays, can be used to measure transgene expression from a vector described herein, thus indicating, e.g., potency of the vector. For example, the HT-22, SK-N-MC, HCN-1A, HCN-2, NT2, SH-SY5y, hNSC11, or ReNcell VM cell lines, or other cell lines that are derived from neuronal or glial cells or progenitors of neuronal or glial cells can be used to assess transgene expression. Once expressed, characteristics of the expressed product (i.e., HuGlyIDUA) can be determined, including determination of the glycosylation and tyrosine sulfation patterns associated with the HuGlyIDUA.

5.2.10. Compositions

Compositions are described comprising a vector encoding a transgene described herein and a suitable carrier. A suitable carrier (e.g., for administration to the CSF, and, for example, to neuronal cells) would be readily selected by one of skill in the art.

5.3 Gene Therapy

Methods are described for the administration of a therapeutically effective amount of a transgene construct to human subjects having MPS I. More particularly, methods for administration of a therapeutically effective amount of a transgene construct to patients having MPS I, in particular, for administration to the CSF are described. In particular embodiments, such methods for administration to the CSF of a therapeutically effective amount of a transgene construct can be used to treat to patients having Hurler syndrome or Hurler-Scheie syndrome.

5.3.1. Target Patient Populations

In certain embodiments, therapeutically effective doses of the recombinant vector are administered to patients diagnosed with MPS I. In specific embodiments, the patients have been diagnosed with Hurler-Scheie syndrome. In specific embodiments, the patients have been diagnosed with Scheie syndrome. In specific embodiments, the patients have been diagnosed with Hurler syndrome.

In certain embodiments, therapeutically effective doses of the recombinant vector are administered to patients diagnosed with severe MPS I. I In certain embodiments, therapeutically effective doses of the recombinant vector are administered to patients diagnosed with attenuated MPS I.

In certain embodiments, therapeutically effective doses of the recombinant vector are administered to patients diagnosed with MPS I who have been identified as responsive to treatment with IDUA, e.g., hIDUA.

In certain embodiments, therapeutically effective doses of the recombinant vector are administered to pediatric patients. In certain embodiments, therapeutically effective doses of the recombinant vector are administered to patients that are less than three years old. In certain embodiments, therapeutically effective doses of the recombinant vector are administered to patients that are aged 2 to 4 years old. In certain embodiments, therapeutically effective doses of the recombinant vector are administered to patients that are aged 3 to 8 years old. In certain embodiments, therapeutically effective doses of the recombinant vector are administered to patients that are aged 8 to 16 years old. In certain embodiments, therapeutically effective doses of the recombinant vector are administered to patients that are aged 6 to 18 years old. In certain embodiments, therapeutically effective doses of the recombinant vector are administered to patients that are 6 years or older. In certain embodiments, therapeutically effective doses of the recombinant vector are administered to patients that are younger than 3 years of age. In certain embodiments, therapeutically effective doses of the recombinant vector are administered to patients that are 4 months or older but younger than 9 months. In certain embodiments, therapeutically effective doses of the recombinant vector are administered to patients that are 9 months or older but younger than 18 months. In certain embodiments, therapeutically effective doses of the recombinant vector are administered to patients that are 18 months or older but younger than 3 years. In certain embodiments, therapeutically effective doses of the recombinant vector are administered to patients that are more than 10 years old. In certain embodiments, therapeutically effective doses of the recombinant vector are administered to adolescent patients. In certain embodiments, therapeutically effective doses of the recombinant vector are administered to adult patients.

In certain embodiments, therapeutically effective doses of the recombinant vector are administered to patients diagnosed with MPS I who have been identified as responsive to treatment with IDUA, e.g., hIDUA, injected into the CSF prior to treatment with gene therapy.

5.3.2. Dosage and Mode of Administration

In certain embodiments, therapeutically effective doses of the recombinant vector are administered to the CSF via intrathecal administration (i.e., injection into the subarachnoid space so that the recombinant vectors distribute through the CSF and transduce cells in the CNS). This can be accomplished in a number of ways—e.g., by intracranial (cistemal or ventricular) injection, or injection into the lumbar cistern. In certain embodiments, intrathecal administration is performed via intracisternal (IC) injection (e.g., into the cisterna magna). In specific embodiments, intracisternal injection is performed by CT-guided suboccipital puncture. In specific embodiments, intrathecal injection is performed by lumbar puncture. In specific embodiments, injection into the subarachnoid space is performed by C1-2 puncture when feasible for the patient. In certain embodiments, therapeutically effective doses of the recombinant vector are administered to the CNS via intranasal administration. In certain embodiments, therapeutically effective doses of the recombinant vector are administered to the CNS via intraparenchymal injection. In certain embodiments, intraparenchymal injection is targeted to the striatum. In certain embodiments, intraparenchymal injection is targeted to the white matter. In certain embodiments, therapeutically effective doses of the recombinant vector are administered to the CSF by any means known to the art, for example, by any means disclosed in Hocquemiller et al., 2016, Human Gene Therapy 27(7):478-496, which is hereby incorporated by reference in its entirety.

The recombinant vector should be administered to the CSF at a dose that maintains a CSF concentration of rHuGlyIDUA at a Cₘᵢₙ of at least 9.25 to 277 μg/mL. In certain embodiments, the recombinant vector is administered to the CSF at a dose that maintains a CSF concentration of rHuGlyIDUA at a Cₘᵢₙ of at least 9.25, 16, 46, 92, 185, or 277 μg/mL. In certain embodiments, the recombinant vector is administered to the CSF at a dose that maintains a CSF concentration of rHuGlylDUA at a Cₘᵢₙ of at least 9.25, 16, 46, 92, 185, or 277 μg/mL. In certain embodiments, the recombinant vector is administered to the CSF at a dose that maintains a CSF concentration of rHuGlyIDUA at a Cₘᵢₙ of at least 9.25 μg/mL. In certain embodiments, the recombinant vector is administered to the CSF at a dose that maintains a CSF concentration of rHuGlyIDUA at a Cₘᵢₙ of at least 16 μg/mL. In certain embodiments, the recombinant vector is administered to the CSF at a dose that maintains a CSF concentration of rHuGlyIDUA at a Cₘᵢₙ of at least 46 μg/mL. In certain embodiments, the recombinant vector is administered to the CSF at a dose that maintains a CSF concentration of rHuGlyIDUA at a Cₘᵢₙ of at least 92 μg/mL. In certain embodiments, the recombinant vector is administered to the CSF at a dose that maintains a CSF concentration of rHuGlyIDUA at a Cₘᵢₙ of at least 185 μg/mL. In certain embodiments, the recombinant vector is administered to the CSF at a dose that maintains a CSF concentration of rHuGlyIDUA at a Cₘᵢₙ of at least 277 μg/mL.

In certain embodiments, for pediatric patients, the recombinant vector is administered to the CSF at a dose that maintains 1.00 to 30.00 mg of total rHuGlyIDUA in the CSF. In certain embodiments, for pediatric patients, the recombinant vector is administered to the CSF at a dose that maintains 1.00, 1.74, 5.00, 10.00, 20.00, or 30.00 mg of total rHuGlyIDUA in the CSF. In certain embodiments, for pediatric patients, the recombinant vector is administered to the CSF at a dose that maintains 1.00 mg of total rHuGlyIDUA in the CSF. In certain embodiments, for pediatric patients, the recombinant vector is administered to the CSF at a dose that maintains 1.74 mg of total rHuGlyIDUA in the CSF. In certain embodiments, for pediatric patients, the recombinant vector is administered to the CSF at a dose that maintains 5.00 mg of total rHuGlyIDUA in the CSF. In certain embodiments, for pediatric patients, the recombinant vector is administered to the CSF at a dose that maintains 10.00 mg of total rHuGlyIDUA in the CSF. In certain embodiments, for pediatric patients, the recombinant vector is administered to the CSF at a dose that maintains 20.00 mg of total rHuGlyIDUA in the CSF. In certain embodiments, for pediatric patients, the recombinant vector is administered to the CSF at a dose that maintains 30.00 mg of total rHuGlyIDUA in the CSF.

In certain embodiments, for adult patients, the recombinant vector is administered to the CSF at a dose that maintains 1.29 to 38.88 mg of total rHuGlyIDUA in the CSF. In certain embodiments, for adult patients, the recombinant vector is administered to the CSF at a dose that maintains 1.29, 2.25, 8.40, 12.96, 25.93, or 38.88 mg of total rHuGlyIDUA in the CSF. In certain embodiments, for adult patients, the recombinant vector is administered to the CSF at a dose that maintains 1.29 mg of total rHuGlyIDUA in the CSF. In certain embodiments, for adult patients, the recombinant vector is administered to the CSF at a dose that maintains 2.25 mg of total rHuGlyIDUA in the CSF. In certain embodiments, for adult patients, the recombinant vector is administered to the CSF at a dose that maintains 8.40 mg of total rHuGlyIDUA in the CSF. In certain embodiments, for adult patients, the recombinant vector is administered to the CSF at a dose that maintains 12.96 mg of total rHuGlyIDUA in the CSF. In certain embodiments, for adult patients, the recombinant vector is administered to the CSF at a dose that maintains 25.93 mg of total rHuGlyIDUA in the CSF. In certain embodiments, for adult patients, the recombinant vector is administered to the CSF at a dose that maintains 38.88 mg of total rHuGlyIDUA in the CSF.

For intrathecal administration, therapeutically effective doses of the recombinant vector should be administered to the CSF in an injection volume, preferably up to about 20 mL. A carrier suitable for intrathecal injection, such as Elliotts B Solution, should be used as a vehicle for the recombinant vectors. Elliots B Solution (generic name: sodium chloride, sodium bicarbonate, anhydrous dextrose, magnesium sulfate, potassium chloride, calcium chloride and sodium phosphate) is a sterile, nonpyrogenic, isotonic solution containing no bacteriostatic preservatives and is used as a diluent for intrathecal administration of chemotherapeutics.

CSF concentrations can be monitored by directly measuring the concentration of rHuGlyIDUA in the CSF fluid obtained from occipital or lumbar punctures, or estimated by extrapolation from concentrations of the rHuGlyIDUA detected in the patient's serum. In certain embodiments, 10 ng/mL to 100 ng/mL of rHuGlyIDUA in the serum is indicative of 1 to 30 mg of rHuGlyIDUA in the CSF. In certain embodiments, the recombinant vector is administered to the CSF at a dose that maintains 10 ng/mL to 100 ng/mL of rHuGlyIDUA in the serum.

In certain embodiments, dosages are measured by the number of genome copies administered to the CSF of the patient (e.g., injected via suboccipital puncture or lumbar puncture). In certain embodiments, 1×10¹² to 2×10¹⁴ genome copies are administered. In certain embodiments, 5×10¹² to 2×10¹⁴ genome copies are administered. In specific embodiments, 1×10¹³ to 1×10¹⁴ genome copies are administered. In specific embodiments, 1×10¹³ to 2×10¹³ genome copies are administered. In specific embodiments, 6×10¹³ to 8×10¹³ genome copies are administered.

In certain embodiments, a flat dose of 1×10¹³ genome copies is administered to a pediatric patient. In certain embodiments, a flat dose of 5.6×10¹³ genome copies is administered to a pediatric patient. In certain embodiments, a flat dose of 1×10¹² to 5.6×10¹³ genome copies is administered to a pediatric patient. In certain embodiments, a flat dose of 1×10¹³ to 5.6×10¹³ genome copies is administered to a pediatric patient. In certain embodiments, a flat dose of 2.6×10¹² genome copies is administered to an adult patient. In certain embodiments, a flat dose of 1.3×10¹³ genome copies is administered to an adult patient. In certain embodiments, a flat dose of 1.4×10¹³ genome copies is administered to an adult patient. In certain embodiments, a flat dose of 7.0×10¹³ genome copies is administered to an adult patient. In certain embodiments, a flat dose of 1.4×10¹³ to 7.0×10¹³ genome copies is administered to an adult patient. In certain embodiments, a flat dose of 1×10¹² to 5.6×10¹³ genome copies is administered to an adult patient.

In certain embodiments, dosages are measured by the number of genome copies administered to the CSF of the patient (e.g., injected via suboccipital puncture or lumbar puncture) per gram of brain mass. In certain embodiments, 1×10⁹ to 2×10¹⁰ genome copies per gram of brain mass are administered. In certain embodiments, 5×10⁹ to 2×10¹⁰ genome copies per gram of brain mass are administered. In certain embodiments, 2×10⁹ genome copies per gram of brain mass are administered. In certain embodiments, 1×10¹⁰ genome copies per gram of brain mass are administered. In specific embodiments, 9×10⁹ to 1×10¹⁰ genome copies per gram of brain mass are administered. In specific embodiments, 1×10¹⁰ to 1.5×10¹⁰ genome copies per gram of brain mass are administered. In specific embodiments, 5×1010 to 6×10¹⁰ genome copies per gram of brain mass are administered.

In one embodiment, a non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive promoter.

The rAAV9.hIDUA may be administered IC (by suboccipital injection) as a single flat dose ranging from 1.4×10¹³ GC (1.1×10¹⁰ GC/g brain mass) to 7.0×10¹³ GC (5.6×1010 GC/g brain mass) in a volume of about 5 to 20 ml, or in a volume of about 5 ml or less. In the event the patient has neutralizing antibodies to AAV, doses at the high range may be used. The rAAV9.hIDUA may be administered IC (by suboccipital injection) as a single flat dose ranging from 2.6×10¹² GC (2×10⁹ GC/g brain mass) to 1.3×10¹³ GC (1×10¹⁰ GC/g brain mass) in a volume of about 5 to 20 ml, or in a volume of about 5 ml or less. When the patient is 4 months or older but younger than 9 months, the rAAV9.hIDUA may be administered IC (by suboccipital injection) as a single flat dose ranging from 6.0×10¹² GC (1.0×10¹⁰ GC/g brain mass) to 3.0×10¹³ GC (5×10¹⁰ GC/g brain mass) in a volume of about 5 to 20 ml, or in a volume of about 5 ml or less. When the patient is 9 months or older but younger than 18 months, the rAAV9.hIDUA may be administered IC (by suboccipital injection) as a single flat dose ranging from 1.0×10¹³ GC (1.0×10¹⁰ GC/g brain mass) to 5.0×10¹³ GC (5×10¹⁰ GC/g brain mass) in a volume of about 5 to 20 ml, or in a volume of about 5 ml or less. When the patient is 18 months or older but younger than 3 years, the rAAV9.hIDUA may be administered IC (by suboccipital injection) as a single flat dose ranging from 1.1×10¹³ GC (1.0×10¹⁰ GC/g brain mass) to 5.5×10¹³ GC (5×10¹⁰ GC/g brain mass) in a volume of about 5 to 20 ml, or in a volume of about 5 ml or less.

5.4 Combination Therapies

5.4.1. Co-Therapy with Immune Suppression

While the delivery of rHuGlyIDUA should minimize immune reactions, the clearest potential source of toxicity related to CNS-directed gene therapy is generating immunity against the expressed hIDUA protein in human subjects who are genetically deficient for IDUA and, therefore, potentially not tolerant of the protein and/or the vector used to deliver the transgene. Thus, in a preferred embodiment, it is advisable to co-treat the patient with immune suppression therapy—especially when treating patients with severe disease who have close to zero levels of IDUA (e.g., patients with Hurler syndrome). Immune suppression therapies involving a regimen of tacrolimus or rapamycin (sirolimus) in combination with mycophenolic acid, or other immune suppression regimens used in tissue transplantation procedures can be employed. Such immune suppression treatment may be administered during the course of gene therapy, and in certain embodiments, pre-treatment with immune suppression therapy may be preferred. Immune suppression therapy can be continued subsequent to the gene therapy treatment, based on the judgment of the treating physician, and may thereafter be withdrawn when immune tolerance is induced; e.g., after 180 days.

In certain embodiments, the methods of treatment provided herein are administered with an immune suppression regimen comprising prednisolone, mycophenolic acid, and tacrolimus. In certain embodiments, the methods of treatment provided herein are administered with an immune suppression regimen comprising prednisolone, mycophenolic acid, and rapamycin (sirolimus). In certain embodiments, the methods of treatment provided herein are administered with an immune suppression regimen that does not comprise tacrolimus. In certain embodiments, the methods of treatment provided herein are administered with an immune suppression regimen comprising one or more corticosteroids such as methylprednisolone and/or prednisolone, as well as tacrolimus and/or sirolimus. In certain embodiments, the immune suppression therapy comprises administering a combination of (a) tacrolimus and mycophenolic acid, or (b) rapamycin and mycophenolic acid to said subject before or concurrently with the human IDUA treatment and continuing thereafter. In certain embodiments, the immune suppression therapy is withdrawn after 180 days. In certain embodiments, the immune suppression therapy is withdrawn after 30, 60, 90, 120, 150, or 180 days.

In certain embodiments, tacrolimus is administered at a dose which results in a serum concentration of 5 to 10 ng/mL. In certain embodiments, tacrolimus is administered at a dose which results in a serum concentration of 4 to 8 ng/mL. In certain embodiments, in particular when the patient is younger than 3 years of age, tacrolimus is administered at a dose which results in a serum concentration of 2 to 4 ng/mL. In certain embodiments, MMF is administered at a dose which results in a serum concentration of 2 to 3.5 μg/mL. In certain embodiments, tacrolimus is administered at a dose which results in a serum concentration of 5 to 10 ng/mL and MMF is administered at a dose which results in a serum concentration of 2 to 3.5 μg/mL. In certain embodiments, serum concentration is achieved by titration of tacrolimus and/or MMF after measurement of trough levels of tacrolimus and/or MMF.

In certain embodiments, methylprednisolone is administered at a dose of 10 mg/kg intravenously once. In certain embodiments, prednisolone is administered at a dose of 0.5 mg/kg orally once daily. In certain embodiments, prednisolone is gradually tapered and then discontinued. In certain embodiments, tacrolimus is administered 1 mg by mouth twice daily to maintain a target blood level of 4-8 ng/ml. In certain embodiments, in particular when the patient is younger than 3 years of age, tacrolimus is administered 0.05 mg/kg by mouth twice daily to maintain a target blood level of 2-4 ng/ml. In certain embodiments sirolimus is also administered. The patient may be pre-dosed with sirolimus which is then maintained at a target blood level of 4-8 ng/ml during the regimen. However, in certain embodiments, when the patient is younger than 3 years of age, the patient is preferably pre-dosed with sirolimus which is then maintained at a target blood level of 1-3 ng/ml during the regimen. In certain embodiments, methylprednisolone is administered at a dose of 10 mg/kg intravenously once, prednisolone is administered at a dose of 0.5 mg/kg orally once daily, tacrolimus is administered 0.2 mg/kg by mouth once daily, and sirolimus is administered.

In certain embodiments, rapamycin is administered at a dose of 2 or 4 mg/kg orally once daily. In certain embodiments, MMF is administered at a dose of 25 mg/kg orally twice daily. In certain embodiments, rapamycin is administered at a dose of 2 or 4 mg/kg orally once daily and MMF is administered at a dose of 25 mg/kg orally twice daily. In certain embodiments, rapamycin is administered at a dose which results in a serum concentration of 5 to 15 ng/mL. In certain embodiments, MMF is administered at a dose which results in a serum concentration of 2 to 3.5 μg/mL. In certain embodiments, rapamycin is administered at a dose which results in a serum concentration of 5 to 15 ng/mL and MMF is administered at a dose which results in a serum concentration of 2 to 3.5 μg/mL. In certain embodiments, serum concentration is achieved by titration of rapamycin and/or MMF after measurement of trough levels of rapamycin and/or MMF.

5.4.2. Co-Therapy with Other Treatments, Including Standard of Care

Combinations of administration of the HuGlyIDUA to the CSF accompanied by administration of other available treatments are encompassed by the methods of the invention. The additional treatments may be administered before, concurrently or subsequent to the gene therapy treatment. Available treatments for MPS I that could be combined with the gene therapy of the invention include but are not limited to enzyme replacement therapy (ERT) using laronidase administered systemically or to the CSF; and/or HSCT therapy. In another embodiment, ERT can be administered using the rHuGlyIDUA glycoprotein produced in human cell lines by recombinant DNA technology. Human cell lines that can be used for such recombinant glycoprotein production include but are not limited to HT-22, SK-N-MC, HCN-1A, HCN-2, NT2, SH-SY5y, hNSC11, ReNcell VM, human embryonic kidney 293 cells (HEK293), fibrosarcoma HT-1080, HKB-11, CAP, HuH-7, and retinal cell lines, PER.C6, or RPE to name a few (see, e.g., Dumont et al., 2016, Critical Rev in Biotech 36(6):1110-1122 “Human cell lines for biopharmaceutical manufacturing: history, status, and future perspectives” which is incorporated by reference in its entirety for a review of the human cell lines that could be used for the recombinant production of the rHuGlyIDUA glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation.

5.5 Biomarkers/Sampling/Monitoring Efficacy

Efficacy may be monitored by measuring cognitive function (e.g., prevention or decrease in neurocognitive decline); reductions in biomarkers of disease (such as GAG) in CSF and or serum; and/or increase in IDUA enzyme activity in CSF and/or serum. Signs of inflammation and other safety events may also be monitored.

5.5.1. Disease Markers

In certain embodiments, efficacy of treatment with the recombinant vector is monitored by measuring the level of a disease biomarker in the patient. In certain embodiments, the level of the disease biomarker is measured in the CSF of the patient. In certain embodiments, the level of the disease biomarker is measured in the serum of the patient. In certain embodiments, the level of the disease biomarker is measured in the urine of the patient. In certain embodiments, the disease biomarker is GAG. In certain embodiments, the disease biomarker is IDUA enzyme activity. In certain embodiments, the disease biomarker is inflammation. In certain embodiments, the disease biomarker is a safety event.

5.5.2. Tests for Neurocognitive Function

In certain embodiments, efficacy of treatment with the recombinant vector is monitored by measuring the level of cognitive function in the patient. Cognitive function may be measured by any method known to one of skill in the art. In certain embodiments, cognitive function is measured via a validated instrument for measuring intelligence quotient (IQ). In specific embodiments, IQ is measured by Wechsler Abbreviated Scale of Intelligence, Second Edition (WASI-II). In certain embodiments, cognitive function is measured via a validated instrument for measuring memory. In specific embodiments, memory is measured by Hopkins Verbal Learning Test (HVLT). In certain embodiments, cognitive function is measured via a validated instrument for measuring attention. In specific embodiments, attention is measured by Test Of Variables of Attention (TOVA). In certain embodiments, cognitive function is measured via a validated instrument for measuring one or more of IQ, memory, and attention.

5.5.3. Physical Changes

In certain embodiments, efficacy of treatment with the recombinant vector is monitored by measuring physical characteristics associated with lysosomal storage deficiency in the patient. In certain embodiments, the physical characteristics are storage lesions. In certain embodiments, the physical characteristic is short stature. In certain embodiments, the physical characteristic is coarsened facial features. In certain embodiments, the physical characteristic is obstructive sleep apnea. In certain embodiments, the physical characteristic is hearing impairment. In certain embodiments, the physical characteristic is vision impairment. In specific embodiments, the visual impairment is due to corneal clouding. In certain embodiments, the physical characteristic is hydrocephalus. In certain embodiments, the physical characteristic is spinal cord compression. In certain embodiments, the physical characteristic is hepatosplenomegaly. In certain embodiments, the physical characteristics are bone and joint deformities. In certain embodiments, the physical characteristic is cardiac valve disease. In certain embodiments, the physical characteristics are recurrent upper respiratory infections. In certain embodiments, the physical characteristic is carpal tunnel syndrome. In certain embodiments, the physical characteristic is macroglossia (enlarged tongue). In certain embodiments, the physical characteristic is enlarged vocal cords and/or change in voice. Such physical characteristics may be measured by any method known to one of skill in the art.

TABLE OF SEQUENCES

| SEQ | | |
|---|---|---|
| ID | | |
| NO: | Description | Sequence |
| 1 | Human IDUA | MRPLRPRAAL LALLASLLAA PPVAPAEAPH |
| | amino acid | LVHVDAARAL WPLRRFWRST GFCPPLPHSQ |
| | sequence | ADQYVLSWDQ QLNLAYVGAV PHRGIKQVRT |
| | | HWLLELVTTR GSTGRGLSYN FTHLDGYLDL |
| | | LRENQLLPGF ELMGSASGHF TDFEDKQQVF |
| | | EWKDLVSSLA RRYIGRYGLA HVSKWNFETW |
| | | NEPDHHDFDN VSMTMQGFLN |
| | | YYDACSEGLR AASPALRLGG PGDSFHTPPR |
| | | SPLSWGLLRH CHDGTNFFTG EAGVRLDYIS |
| | | LHRKGARSSI SILEQEKVVA QQIRQLFPKF |
| | | ADTPIYNDEA DPLVGWSLPQ PWRADVTYAA |
| | | MVVKVIAQHQ NLLLANTTSA FPYALLSNDN |
| | | AFLSYHPHPF AQRTLTARFQ VNNTRPPHVQ |
| | | LLRKPVLTAM GLLALLDEEQ |
| | | LWAEVSQAGT VLDSNHTVGV LASAHRPQGP |
| | | ADAWRAAVLI YASDDTRAHP NRSVAVTLRL |
| | | RGVPPGPGLV YVTRYLDNGL CSPDGEWRRL |
| | | GRPVFPTAEQ FRRMRAAEDP VAAAPRPLPA |
| | | GGRLTLRPAL RLPSLLLVHV CARPEKPPGQ |
| | | VTRLRALPLT QGQLVLVWSD EHVGSKCLWT |
| | | YEIQFSQDGK AYTPVSRKPS |
| | | TFNLFVFSPD TGAVSGSYRV RALDYWARPG |
| | | PFSDPVPYLE VPVPRGPPSP GNP |
| 2 | Oligodendrocyte- | MEYQILKMSL CLFILLFLTP GILC |
| | myelin | |
| | glycoprotein | |
| | (hOMG) signal | |
| | peptide | |
| 3 | Cellular repressor | MSVRRGRRPA RPGTRLSWLL CCSALLSPAA G |
| | of E1A-stimulated | |
| | genes 2 (hCREG2) | |
| | signal peptide | |
| 4 | V-set and | MEQRNRLGAL GYLPPLLLHA LLLFVADA |
| | transmembrane | |
| | domain containing | |
| | 2B (hVSTM2B) | |
| | signal peptide | |
| 5 | Protocadherin | MVFSRRGGLG ARDLLLWLLL LAAWEVGSG |
| | alpha-1 | |
| | (hPCADHA1) | |
| | signal peptide | |
| 6 | FAM19A1 | MAMVSAMSWV LYLWISACA |
| | (TAFA1) signal | |
| | peptide | |
| 7 | VEGF-A signal | MNFLLSWVHW SLALLLYLHH AKWSQA |
| | peptide | |
| 8 | Fibulin-1 signal | MERAAPSRRV PLPLLLLGGL ALLAAGVDA |
| | peptide | |
| 9 | Vitronectin signal | MAPLRPLLIL ALLAWVALA |
| | peptide | |
| 10 | Complement | MRLLAKIICL MLWAICVA |
| | Factor H signal | |
| | peptide | |
| 11 | Opticin signal | MRLLAFLSLL ALVLQETGT |
| | peptide | |
| 12 | Albumin signal | MKWVTFISLL FLFSSAYS |
| | peptide | |
| 13 | Chymotrypsinogen | MAFLWLLSCW ALLGTTFG |
| | signal peptide | |
| 14 | Interleukin-2 signal | MYRMQLLSCI ALILALVTNS |
| | peptide | |
| 15 | Trypsinogen-2 | MNLLLILTFV AAAVA |
| | signal peptide | |
| 16 | AAV1 | MAADGYLPDWLEDNLSEGIREWWDLKPGAPKPKANQQK |
| | | QDDGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPLGLVEEGAKTAPGKKRPVEQSPQE |
| | | PDSSSGIGKTGQQPAKKRLNFGQTGDSESVPDPQPLGE |
| | | PPATPAAVGPTTMASGGGAPMADNNEGADGVGNASGNW |
| | | HCDSTWLGDRVITTSTRTWALPTYNNHLYKQISSASTG |
| | | ASNDNHYFGYSTPWGYFDFNRFHCHFSPRDWQRLINNN |
| | | WGFRPKRLNFKLFNIQVKEVTTNDGVTTIANNLTSTVQ |
| | | VFSDSEYQLPYVLGSAHQGCLPPFPADVFMIPQYGYLT |
| | | LNNGSQAVGRSSFYCLEYFPSQMLRTGNNFTFSYTFEE |
| | | VPFHSSYAHSQSLDRLMNPLIDQYLYYLNRTQNQSGSA |
| | | QNKDLLFSRGSPAGMSVQPKNWLPGPCYRQQRVSKTKT |
| | | DNNNSNFTWTGASKYNLNGRESIINPGTAMASHKDDED |
| | | KFFPMSGVMIFGKESAGASNTALDNVMITDEEEIKATN |
| | | PVATERFGTVAVNFQSSSTDPATGDVHAMGALPGMVWQ |
| | | DRDVYLQGPIWAKIPHTDGHFHPSPLMGGFGLKNPPPQ |
| | | ILIKNTPVPANPPAEFSATKFASFITQYSTGQVSVEIE |
| | | WELQKENSKRWNPEVQYTSNYAKSANVDFTVDNNGLYT |
| | | EPRPIGTRYLTRPL |
| 17 | AAV2 | MAADGYLPDWLEDTLSEGIRQWWKLKPGPPPPKPAERH |
| | | KDDSRGLVLPGYKYLGPFNGLDKGEPVNEADAAALEHD |
| | | KAYDRQLDSGDNPYLKYNHADAEFQERLKEDTSFGGNL |
| | | GRAVFQAKKRVLEPLGLVEEPVKTAPGKKRPVEHSPVE |
| | | PDSSSGTGKAGQQPARKRLNFGQTGDADSVPDPQPLGQ |
| | | PPAAPSGLGTNTMATGSGAPMADNNEGADGVGNSSGNW |
| | | HCDSTWMGDRVITTSTRTWALPTYNNHLYKQISSQSGA |
| | | SNDNHYFGYSTPWGYFDFNRFHCHFSPRDWQRLINNNW |
| | | GFRPKRLNFKLFNIQVKEVTQNDGTTTIANNLTSTVQV |
| | | FTDSEYQLPYVLGSAHQGCLPPFPADVFMVPQYGYLTL |
| | | NNGSQAVGRSSFYCLEYFPSQMLRTGNNFTFSYTFEDV |
| | | PFHSSYAHSQSLDRLMNPLIDQYLYYLSRTNTPSGTTT |
| | | QSRLQFSQAGASDIRDQSRNWLPGPCYRQQRVSKTSAD |
| | | NNNSEYSWTGATKYHLNGRDSLVNPGPAMASHKDDEEK |
| | | FFPQSGVLIFGKQGSEKTNVDIEKVMITDEEEIRTTNP |
| | | VATEQYGSVSTNLQRGNRQAATADVNTQGVLPGMVWQD |
| | | RDVYLQGPIWAKIPHTDGHFHPSPLMGGFGLKHPPPQI |
| | | LIKNTPVPANPSTTFSAAKFASFITQYSTGQVSVEIEW |
| | | ELQKENSKRWNPEIQYTSNYNKSVNVDFTVDTNGVYSE |
| | | PRPIGTRYLTRNL |
| 18 | AAV3-3 | MAADGYLPDWLEDNLSEGIREWWALKPGVPQPKANQQH |
| | | QDNRRGLVLPGYKYLGPGNGLDKGEPVNEADAAALEHD |
| | | KAYDQQLKAGDNPYLKYNHADAEFQERLQEDTSFGGNL |
| | | GRAVFQAKKRILEPLGLVEEAAKTAPGKKGAVDQSPQE |
| | | PDSSSGVGKSGKQPARKRLNFGQTGDSESVPDPQPLGE |
| | | PPAAPTSLGSNTMASGGGAPMADNNEGADGVGNSSGNW |
| | | HCDSQWLGDRVITTSTRTWALPTYNNHLYKQISSQSGA |
| | | SNDNHYFGYSTPWGYFDFNRFHCHFSPRDWQRLINNNW |
| | | GFRPKKLSFKLFNIQVRGVTQNDGTTTIANNLTSTVQV |
| | | FTDSEYQLPYVLGSAHQGCLPPFPADVFMVPQYGYLTL |
| | | NNGSQAVGRSSFYCLEYFPSQMLRTGNNFQFSYTFEDV |
| | | PFHSSYAHSQSLDRLMNPLIDQYLYYLNRTQGTTSGTT |
| | | NQSRLLFSQAGPQSMSLQARNWLPGPCYRQQRLSKTAN |
| | | DNNNSNFPWTAASKYHLNGRDSLVNPGPAMASHKDDEE |
| | | KFFPMHGNLIFGKEGTTASNAELDNVMITDEEEIRTTN |
| | | PVATEQYGTVANNLQSSNTAPTTGTVNHQGALPGMVWQ |
| | | DRDVYLQGPIWAKIPHTDGHFHPSPLMGGFGLKHPPPQ |
| | | IMIKNTPVPANPPTTFSPAKFASFITQYSTGQVSVEIE |
| | | WELQKENSKRWNPEIQYTSNYNKSVNVDFTVDTNGVYS |
| | | EPRPIGTRYLTRNL |
| 19 | AAV4-4 | MTDGYLPDWLEDNLSEGVREWWALQPGAPKPKANQQHQ |
| | | DNARGLVLPGYKYLGPGNGLDKGEPVNAADAAALEHDK |
| | | AYDQQLKAGDNPYLKYNHADAEFQQRLQGDTSFGGNLG |
| | | RAVFQAKKRVLEPLGLVEQAGETAPGKKRPLTESPQQP |
| | | DSSTGIGKKGKQPAKKKLVFEDETGAGDGPPEGSTSGA |
| | | MSDDSEMRAAAGGAAVEGGQGADGVGNASGDWHCDSTW |
| | | SEGHVTTTSTRTWVLPTYNNHLYKRLGESLQSNTYNGF |
| | | STPWGYFDFNRFHCHFSPRDWQRLINNNWGMRPKAMRV |
| | | KIFNIQVKEVTTSNGETTVANNLTSTVQIFADSSYELP |
| | | YVMDAGQEGSLPPFPNDVFMVPQYGYCGLVTGNTSQQQ |
| | | TDRNAFYCLEYFPSQMLRTGNNFEITYSFEKVPFHSMY |
| | | AHSQSLDRLMNPLIDQYLWGLQSTTTGTTLNAGTATTN |
| | | FTKLRPTNFSNFKKNWLPGPSIKQQGFSKTANQNYKIP |
| | | ATGSDSLIKYETHSTLDGRWSALTPGPPMATAGPADSK |
| | | FSNSQLIFAGPKQNGNTATVPGTLIFTSEEELAATNAT |
| | | DTDMWGNLPGGDQSNSNLPTVDRLTALGAVPGMVWQNR |
| | | DIYYQGPIWAKIPHTDGHFHPSPLIGGFGLKHPPPQIF |
| | | IKNTPVPANPATTFSSTPVNSFITQYSTGQVSVQIDWE |
| | | IQKERSKRWNPEVQFTSNYGQQNSLLWAPDAAGKYTEP |
| | | RAIGTRYLTHHL |
| 20 | AAV5 | MSFVDHPPDWLEEVGEGLREFLGLEAGPPKPKPNQQHQ |
| | | DQARGLVLPGYNYLGPGNGLDRGEPVNRADEVAREHDI |
| | | SYNEQLEAGDNPYLKYNHADAEFQEKLADDTSFGGNLG |
| | | KAVFQAKKRVLEPFGLVEEGAKTAPTGKRIDDHFPKRK |
| | | KARTEEDSKPSTSSDAEAGPSGSQQLQIPAQPASSLGA |
| | | DTMSAGGGGPLGDNNQGADGVGNASGDWHCDSTWMGDR |
| | | VVTKSTRTWVLPSYNNHQYREIKSGSVDGSNANAYFGY |
| | | STPWGYFDFNRFHSHWSPRDWQRLINNYWGFRPRSLRV |
| | | KIFNIQVKEVTVQDSTTTIANNLTSTVQVFTDDDYQLP |
| | | YVVGNGTEGCLPAFPPQVFTLPQYGYATLNRDNTENPT |
| | | ERSSFFCLEYFPSKMLRTGNNFEFTYNFEEVPFHSSFA |
| | | PSQNLFKLANPLVDQYLYRFVSTNNTGGVQFNKNLAGR |
| | | YANTYKNWFPGPMGRTQGWNLGSGVNRASVSAFATTNR |
| | | MELEGASYQVPPQPNGMTNNLQGSNTYALENTMIFNSQ |
| | | PANPGTTATYLEGNMLITSESETQPVNRVAYNVGGQMA |
| | | TNNQSSTTAPATGTYNLQEIVPGSVWMERDVYLQGPIW |
| | | AKIPETGAHFHPSPAMGGFGLKHPPPMMLIKNTPVPGN |
| | | ITSFSDVPVSSFITQYSTGQVTVEMEWELKKENSKRWN |
| | | PEIQYTNNYNDPQFVDFAPDSTGEYRTTRPIGTRYLTR |
| | | PL |
| 21 | AAV6 | MAADGYLPDWLEDNLSEGIREWWDLKPGAPKPKANQQK |
| | | QDDGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPFGLVEEGAKTAPGKKRPVEQSPQE |
| | | PDSSSGIGKTGQQPAKKRLNFGQTGDSESVPDPQPLGE |
| | | PPATPAAVGPTTMASGGGAPMADNNEGADGVGNASGNW |
| | | HCDSTWLGDRVITTSTRTWALPTYNNHLYKQISSASTG |
| | | ASNDNHYFGYSTPWGYFDFNRFHCHFSPRDWQRLINNN |
| | | WGFRPKRLNFKLFNIQVKEVTTNDGVTTIANNLTSTVQ |
| | | VFSDSEYQLPYVLGSAHQGCLPPFPADVFMIPQYGYLT |
| | | LNNGSQAVGRSSFYCLEYFPSQMLRTGNNFTFSYTFED |
| | | VPFHSSYAHSQSLDRLMNPLIDQYLYYLNRTQNQSGSA |
| | | QNKDLLFSRGSPAGMSVQPKNWLPGPCYRQQRVSKTKT |
| | | DNNNSNFTWTGASKYNLNGRESIINPGTAMASHKDDKD |
| | | KFFPMSGVMIFGKESAGASNTALDNVMITDEEEIKATN |
| | | PVATERFGTVAVNLQSSSTDPATGDVHVMGALPGMVWQ |
| | | DRDVYLQGPIWAKIPHTDGHFHPSPLMGGFGLKHPPPQ |
| | | ILIKNTPVPANPPAEFSATKFASFITQYSTGQVSVEIE |
| | | WELQKENSKRWNPEVQYTSNYAKSANVDFTVDNNGLYT |
| | | EPRPIGTRYLTRPL |
| 22 | AAV7 | MAADGYLPDWLEDNLSEGIREWWDLKPGAPKPKANQQK |
| | | QDNGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPLGLVEEGAKTAPAKKRPVEPSPQR |
| | | SPDSSTGIGKKGQQPARKRLNFGQTGDSESVPDPQPLG |
| | | EPPAAPSSVGSGTVAAGGGAPMADNNEGADGVGNASGN |
| | | WHCDSTWLGDRVITTSTRTWALPTYNNHLYKQISSETA |
| | | GSTNDNTYFGYSTPWGYFDFNRFHCHFSPRDWQRLINN |
| | | NWGFRPKKLRFKLFNIQVKEVTTNDGVTTIANNLTSTI |
| | | QVFSDSEYQLPYVLGSAHQGCLPPFPADVFMIPQYGYL |
| | | TLNNGSQSVGRSSFYCLEYFPSQMLRTGNNFEFSYSFE |
| | | DVPFHSSYAHSQSLDRLMNPLIDQYLYYLARTQSNPGG |
| | | TAGNRELQFYQGGPSTMAEQAKNWLPGPCFRQQRVSKT |
| | | LDQNNNSNFAWTGATKYHLNGRNSLVNPGVAMATHKDD |
| | | EDRFFPSSGVLIFGKTGATNKTTLENVLMTNEEEIRPT |
| | | NPVATEEYGIVSSNLQAANTAAQTQVVNNQGALPGMVW |
| | | QNRDVYLQGPIWAKIPHTDGNFHPSPLMGGFGLKHPPP |
| | | QILIKNTPVPANPPEVFTPAKFASFITQYSTGQVSVEI |
| | | EWELQKENSKRWNPEIQYTSNFEKQTGVDFAVDSQGVY |
| | | SEPRPIGTRYLTRNL |
| 23 | AAV8 | MAADGYLPDWLEDNLSEGIREWWALKPGAPKPKANQQK |
| | | QDDGRGLVLPGYKYLGPFNGLDKGEPVNAADAAALEHD |
| | | KAYDQQLQAGDNPYLRYNHADAEFQERLQEDTSFGGNL |
| | | GRAVFQAKKRVLEPLGLVEEGAKTAPGKKRPVEPSPQR |
| | | SPDSSTGIGKKGQQPARKRLNFGQTGDSESVPDPQPLG |
| | | EPPAAPSGVGPNTMAAGGGAPMADNNEGADGVGSSSGN |
| | | WHCDSTWLGDRVITTSTRTWALPTYNNHLYKQISNGTS |
| | | GGATNDNTYFGYSTPWGYFDFNRFHCHFSPRDWQRLIN |
| | | NNWGFRPKRLSFKLFNIQVKEVTQNEGTKTIANNLTST |
| | | IQVFTDSEYQLPYVLGSAHQGCLPPFPADVFMIPQYGY |
| | | LTLNNGSQAVGRSSFYCLEYFPSQMLRTGNNFQFTYTF |
| | | EDVPFHSSYAHSQSLDRLMNPLIDQYLYYLSRTQTTGG |
| | | TANTQTLGFSQGGPNTMANQAKNWLPGPCYRQQRVSTT |
| | | TGQNNNSNFAWTAGTKYHLNGRNSLANPGIAMATHKDD |
| | | EERFFPSNGILIFGKQNAARDNADYSDVMLTSEEEIKT |
| | | TNPVATEEYGIVADNLQQQNTAPQIGTVNSQGALPGMV |
| | | WQNRDVYLQGPIWAKIPHTDGNFHPSPLMGGFGLKHPP |
| | | PQILIKNTPVPADPPTTFNQSKLNSFITQYSTGQVSVE |
| | | IEWELQKENSKRWNPEIQYTSNYYKSTSVDFAVNTEGV |
| | | YSEPRPIGTRYLTRNL |
| 24 | hu31 | MAADGYLPDWLEDTLSEGIRQWWKLKPGPPPPKPAERH |
| | | KDDSRGLVLPGYKYLGPGNGLDKGEPVNAADAAALEHD |
| | | KAYDQQLKAGDNPYLKYNHADAEFQERLKEDTSFGGNL |
| | | GRAVFQAKKRLLEPLGLVEEAAKTAPGKKRPVEQSPQE |
| | | PDSSAGIGKSGSQPAKKKLNFGQTGDTESVPDPQPIGE |
| | | PPAAPSGVGSLTMASGGGAPVADNNEGADGVGSSSGNW |
| | | HCDSQWLGDRVITTSTRTWALPTYNNHLYKQISNSTSG |
| | | GSSNDNAYFGYSTPWGYFDFNRFHCHFSPRDWQRLINN |
| | | NWGFRPKRLNFKLFNIQVKEVTDNNGVKTIANNLTSTV |
| | | QVFTDSDYQLPYVLGSAHEGCLPPFPADVFMIPQYGYL |
| | | TLNDGGQAVGRSSFYCLEYFPSQMLRTGNNFQFSYEFE |
| | | NVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGED |
| | | RFFPLSGSLIFGKQGTGRDNVDADKVMITNEEEIKTTN |
| | | PVATESYGQVATNHQSAQAQAQTGWVQNQGILPGMVWQ |
| | | DRDVYLQGPIWAKIPHTDGNFHPSPLMGGFGMKHPPPQ |
| | | ILIKNTPVPADPPTAFNKDKLNSFITQYSTGQVSVEIE |
| | | WELQKENSKRWNPEIQYTSNYYKSNNVEFAVSTEGVYS |
| | | EPRPIGTRYLTRNL |
| 25 | hu32 | MAADGYLPDWLEDTLSEGIRQWWKLKPGPPPPKPAERH |
| | | KDDSRGLVLPGYKYLGPGNGLDKGEPVNAADAAALEHD |
| | | KAYDQQLKAGDNPYLKYNHADAEFQERLKEDTSFGGNL |
| | | GRAVFQAKKRLLEPLGLVEEAAKTAPGKKRPVEQSPQE |
| | | PDSSAGIGKSGSQPAKKKLNFGQTGDTESVPDPQPIGE |
| | | PPAAPSGVGSLTMASGGGAPVADNNEGADGVGSSSGNW |
| | | HCDSQWLGDRVITTSTRTWALPTYNNHLYKQISNSTSG |
| | | GSSNDNAYFGYSTPWGYFDFNRFHCHFSPRDWQRLINN |
| | | NWGFRPKRLNFKLFNIQVKEVTDNNGVKTIANNLTSTV |
| | | QVFTDSDYQLPYVLGSAHEGCLPPFPADVFMIPQYGYL |
| | | TLNDGSQAVGRSSFYCLEYFPSQMLRTGNNFQFSYEFE |
| | | NVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGED |
| | | RFFPLSGSLIFGKQGTGRDNVDADKVMITNEEEIKTTN |
| | | PVATESYGQVATNHQSAQAQAQTGWVQNQGILPGMVWQ |
| | | DRDVYLQGPIWAKIPHTDGNFHPSPLMGGFGMKHPPPQ |
| | | ILIKNTPVPADPPTAFNKDKLNSFITQYSTGQVSVEIE |
| | | WELQKENSKRWNPEIQYTSNYYKSNNVEFAVNTEGVYS |
| | | EPRPIGTRYLTRNL |
| 26 | AAV9 | MAADGYLPDWLEDNLSEGIREWWALKPGAPQPKANQQH |
| | | QDNARGLVLPGYKYLGPGNGLDKGEPVNAADAAALEHD |
| | | KAYDQQLKAGDNPYLKYNHADAEFQERLKEDTSFGGNL |
| | | GRAVFQAKKRLLEPLGLVEEAAKTAPGKKRPVEQSPQE |
| | | PDSSAGIGKSGAQPAKKRLNFGQTGDTESVPDPQPIGE |
| | | PPAAPSGVGSLTMASGGGAPVADNNEGADGVGSSSGNW |
| | | HCDSQWLGDRVITTSTRTWALPTYNNHLYKQISNSTSG |
| | | GSSNDNAYFGYSTPWGYFDFNRFHCHFSPRDWQRLINN |
| | | NWGFRPKRLNFKLFNIQVKEVTDNNGVKTIANNLTSTV |
| | | QVFTDSDYQLPYVLGSAHEGCLPPFPADVFMIPQYGYL |
| | | TLNDGSQAVGRSSFYCLEYFPSQMLRTGNNFQFSYEFE |
| | | NVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGED |
| | | RFFPLSGSLIFGKQGTGRDNVDADKVMITNEEEIKTTN |
| | | PVATESYGQVATNHQSAQAQAQTGWVQNQGILPGMVWQ |
| | | DRDVYLQGPIWAKIPHTDGNFHPSPLMGGFGMKHPPPQ |
| | | ILIKNTPVPADPPTAFNKDKLNSFITQYSTGQVSVEIE |
| | | WELQKENSKRWNPEIQYTSNYYKSNNVEFAVNTEGVYS |
| | | EPRPIGTRYLTRNL |

### 6. EXAMPLES

### 6.1 Example 1: hIDUA cDNA

A hIDUA cDNA-based vector is constructed comprising a transgene comprising hIDUA (SEQ ID NO:1). The transgene also comprises nucleic acids comprising a signal peptide chosen from the group listed in Table 1. Optionally, the vector additionally comprises a promoter.

### 6.2 Example 2: Substituted hIDUA cDNAs

A hIDUA cDNA-based vector is constructed comprising a transgene comprising hIDUA having amino acid substitutions, deletions, or additions compared to the hIDUA sequence of SEQ ID NO: 1, e.g., including but not limited to amino acid substitutions selected from corresponding non-conserved residues in orthologs of IDUA shown in FIG. 2, with the proviso that such mutations do not include any that have been identified in severe, severe-intermediate, intermediate, or attenuated MPS I phenotypes shown in FIG. 3 (from, Saito et al., 2014, Mol Genet Metab 111:107-112, Table 1 listing 57 MPS I mutations, which is incorporated by reference herein in its entirety); or reported by Venturi et al., 2002, Human Mutation #522 Online (“Venturi 2002”), or Bertola et al., 2011 Human Mutation 32:E2189-E2210 (“Bertola 2011”), each of which is incorporated by reference herein in its entirety. The transgene also comprises nucleic acids comprising a signal peptide chosen from the group listed in Table 1. Optionally, the vector additionally comprises a promoter.

### 6.3 Example 3: Treatment of MPS I in Animals Models with hIDUA or Substituted hIDUA

An hIDUA cDNA-based vector is deemed useful for treatment of MPS I when expressed as a transgene. An animal model for MPS I, for example an animal model described in Clarke et al., 1997, Hum Mol Genet 6(4):503-511 (mice), Haskins et al., 1979, Pediatr Res 13(11): 1294-97 (the domestic shorthair cat), Menon et al., 1992, Genomics 14(3):763-768 (dog), or Shull et al., 1982, Am J Pathol 109(2):244-248 (dog), is administered a recombinant vector that encodes hIDUA intrathecally at a dose sufficient to deliver and maintain a concentration of the transgene product at a Cₘᵢₙ of at least 9.25 μg/mL in the CSF of the animal. Following treatment, the animal is evaluated for improvement in symptoms consistent with the disease in the particular animal model.

### 6.4 Example 4: Treatment of MPS I with hIDUA or Substituted hIDUA

An hIDUA cDNA-based vector is deemed useful for treatment of MPS I when expressed as a transgene. A subject presenting with MPS I is administered a cDNA-based vector that encodes hIDUA intrathecally at a dose sufficient to deliver and maintain a concentration of the transgene product at a Cₘᵢₙ of at least 9.25 μg/mL in the CSF. Following treatment, the subject is evaluated for improvement in symptoms of MPS I. Prior to, concurrently with, or after administration of the cDNA-based vector that encodes hIDUA, the patient is administered immunosuppression therapy comprising rapamycin, MMF, and prednisolone.

### 6.5 Example 5: Clinical Protocol Treatment of MPS I

The following example sets out a protocol that may be used to treat human subjects with a rAAV9.hIDUA vector to treat MPS I.

Patient Population.

Patients to be treated may include males or females who have:
a diagnosis of MPS I confirmed by enzyme activity, as measured in plasma, fibroblasts, or leukocytes.
early-stage neurocognitive deficit due to MPS I, defined as either of the following, if not explainable by any other neurologic or psychiatric factors:
   A score of ≥1 standard deviation below mean on IQ testing or in 1 domain of neuropsychological function (verbal comprehension, memory, attention, or perceptual reasoning).
   Documented historical evidence (medical records) of a decline of >1 standard deviation on sequential testing.

Patients can include those who have on a stable regimen of ERT (e.g., ALDURAZYME [laronidase] IV). Females of childbearing potential should have a negative serum pregnancy test on the day of treatment. Sexually active subjects (both female and male) should use a medically accepted method of barrier contraception (e.g., condom, diaphragm, or abstinence) until 24 weeks after vector administration. Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC.

Patients having any condition that the treating physician believes would not be appropriate for immunosuppressive therapy should not receive treatment (e.g., absolute neutrophil count <1.3×10³/μL, platelet count <100×10³/μL, and hemoglobin <12 g/dL [male] or <10 g/dL [female]). An alternative immune suppression regimen should be used on any patient who has any history of a hypersensitivity reaction to sirolimus, MMF, or prednisolone.

Patients with a history of lymphoma or another cancer, other than squamous cell or basal cell carcinoma of the skin, should not be treated unless in full remission for at least 3 months before treatment.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome and a fractionated bilirubin that shows conjugated bilirubin <35% of total bilirubin.

Patients with a history of infectious disease or substance abuse may not be candidates for treatment. For example, a history of human immunodeficiency virus (HIV)-positive test, history of active or recurrent hepatitis B or hepatitis C, or positive screening tests for hepatitis B, hepatitis C, or HIV; a history of alcohol or substance abuse within 1 year before treatment.

In one embodiment, the patients are adult patients. In another embodiment, the patients are pediatric patients.

Treatments Administered—Pre-treatment with Immunosuppressive Therapy.

Prior to gene therapy, the patient should be treated with an immunosuppressive therapy to prevent immune responses to the transgene and/or AAV capsid. Such immunosuppressive therapy includes prednisolone (60 mg PO QD Days −2 to 8), MMF (1 g PO BID Days −2 to 60), and sirolimus (6 mg PO Day −2 then 2 mg QD from Day-1 until Week 48). Sirolimus dose adjustments are made to maintain whole blood trough concentrations within 16-24 ng/mL. In most subjects, dose adjustments can be based on the equation: new dose=current dose×(target concentration/current concentration). Subjects should continue on the new maintenance dose for at least 7-14 days before further dosage adjustment with concentration monitoring. If neutropenia develops (absolute neutrophil count <1.3×10³/μL), MMF dosing should be interrupted or the dose reduced.

Gene Therapy.

A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

The rAAV9.hIDUA is administered as a single flat dose by IC administration: either a low dose of 1.4×10¹³ GC (1.1×10¹⁰ GC/g brain mass), or a high dose of 7.0×10¹³ GC (5.6×10¹⁰ GC/g brain mass) can be used in a volume of about 5 to 20 ml. In the event the patient has neutralizing antibodies to AAV, the high dose may be used.

For administration of rAAV9.IDUA, the subject is put under general anesthesia. A lumbar puncture is performed, first to remove 5 cc of CSF and subsequently to inject contrast IT to aid visualization of the cisterna magna. CT (with contrast) is utilized to guide needle insertion and administration of the selected dose of rAAV9.IDUA into the suboccipital space.

### 6.6 Example 6: Clinical Protocol Treatment of MPS I

The following example sets out a protocol that may be used to treat human subjects with a rAAV9.hIDUA vector to treat MPS I.

Patient Population.

Patients to be treated may include males or females 6 years of age or older who have:
a diagnosis of MPS I confirmed by enzyme activity, as measured in plasma, fibroblasts, or leukocytes (this includes those who may have previously received HSCT or have previously or are currently receiving laronidase treatment).
early-stage neurocognitive deficit due to MPS I, defined as either of the following, if not explainable by any other neurologic or psychiatric factors:
   A score of ≥1 standard deviation below mean on IQ testing or in 1 domain of neuropsychological function (verbal comprehension, attention, or perceptual reasoning).
   A decline of >1 standard deviation on sequential testing.

Patients should have sufficient auditory and visual capacity, with or without aids, to complete the required protocol testing and willing to be compliant with wearing the aid, if applicable, on testing days.

Females of childbearing potential should have a negative serum pregnancy test on the day of treatment. All sexually active subjects must be willing to use a medically accepted method of barrier contraception from the screening visit until 24 weeks after vector administration. Sexually active females must be willing to use an effective method of birth control from the screening visit until 12 weeks after the last dose of sirolimus, whichever is later. Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC. Patients having any condition that the treating physician believes would not be appropriate for immunosuppressive therapy should not receive treatment (e.g., absolute neutrophil count <1.3×10³/μL, platelet count <100×10³/μL, and hemoglobin <12 g/dL [male] or <10 g/dL [female]). An alternative immune suppression regimen should be used on any patient who has any history of a hypersensitivity reaction to sirolimus, MMF, or prednisolone.

Patients with a history of lymphoma or another cancer, other than squamous cell or basal cell carcinoma of the skin, should not be treated unless in full remission for at least 3 months before treatment.

Patients with uncontrolled hypertension (systolic BP >180 mmHg, diastolic BP >100 mmHg) despite maximal medical treatment should not be treated.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome and a fractionated bilirubin that shows conjugated bilirubin <35% of total bilirubin.

Patients with a history of infectious disease or substance abuse may not be candidates for treatment. For example, a history of human immunodeficiency virus (HIV) or hepatitis B or hepatitis C virus infection, or positive screening tests for hepatitis B surface antigen or hepatitis B core antibody, or hepatitis C or HIV antibodies; a history of alcohol or substance abuse within 1 year before screening.

Patients who have received any investigational product within 30 days or 5 half-lives before, whichever is longer, should not be treated except patients administered IT laronidase, which can be administered at any time prior.

Patients who are pregnant, less than six weeks postpartum, breastfeeding at screening, or planning to become pregnant at any time through Week 52 should not be treated.

Patients with a clinically significant ECG abnormality that would compromise the subject's safety should not be treated. Patients with a serious or unstable medical or psychological condition that would compromise the subject's safety should not be treated.

Treatments Administered—Pre-treatment with Immunosuppressive Therapy.

Prior to gene therapy, the patient should be treated with an immunosuppressive therapy to prevent immune responses to the transgene and/or AAV capsid. Such immunosuppressive therapy includes prednisolone (60 mg PO QD Days −2 to 8), MMF (1 g PO BID Days −2 to 60), and sirolimus (6 mg Po Day −2 then 2 mg QD from Day-1 until Week 48). Sirolimus dose adjustments are made to maintain whole blood trough concentrations within 16-24 ng/mL. In most subjects, dose adjustments can be based on the equation: new dose=current dose x (target concentration/current concentration). Subjects should continue on the new maintenance dose for at least 7-14 days before further dosage adjustment with concentration monitoring.

The underlying principle for the immunosuppression regimen is to administer corticosteroids to fully suppress immunity—starting with an IV methylprednisolone to load the dose, and following with oral prednisolone that is gradually tapered down so that the patient is off steroids by week 12. The corticosteroid treatment is supplemented by tacrolimus (for 24 weeks) and/or sirolimus (for 12 weeks), and can be further supplemented with MMF. When using both tacrolimus and sirolimus, the dose of each should be a low dose adjusted to maintain a blood trough level of 4-8 ng/ml. If only one of the agents is used, the label dose (higher dose) should be employed; e.g., tacrolimus at 0.15-0.20 mg/kg/day given as two divided doses every 12 hours; and sirolimus at 1 mg/m²/day; the loading dose should be 3 mg/m². If MMF is added to the regimen, the dose for tacrolimus and/or sirolimus can be maintained since the mechanisms of action differ.

Gene Therapy. A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

The rAAV9.hIDUA is administered as a single flat dose by IC administration: either a dose of ≥2×10⁹ GC/g brain mass (2.6×10¹² GC), or a dose of 1×10¹⁰ GC/g brain mass (1.3×10¹³ GC). The dose can be in a volume of about 5 to 20 ml.

For administration of rAAV9.IDUA, the subject is put under general anesthesia.

### 6.7 Example 7: Clinical Protocol Treatment of MPS I

The following example sets out a protocol that may be used to treat human subjects with a rAAV9.hIDUA vector to treat MPS I.

Patient Population.

Patients to be treated may include males or females who have:
a diagnosis of MPS I confirmed by enzyme activity, as measured in plasma, fibroblasts, or leukocytes (this includes those who may have previously or currently received HSCT or laronidase treatment).
early-stage neurocognitive deficit due to MPS I, defined as either of the following, if not explainable by any other neurologic or psychiatric factors:
   A score of ≥1 standard deviation below mean on IQ testing or in 1 domain of neuropsychological function (verbal comprehension, attention, or perceptual reasoning).
   A decline of ≥1 standard deviation on sequential testing.

Patients should have sufficient auditory and visual capacity, with or without aids, to complete the required protocol testing and willing to be compliant with wearing the aid, if applicable, on testing days.

Females of childbearing potential should have a negative serum pregnancy test on the day of treatment. All sexually active subjects must be willing to use a medically accepted method of barrier contraception from the screening visit until 24 weeks after vector administration. Sexually active females must be willing to use an effective method of birth control from the screening visit until 12 weeks after the last dose of sirolimus, whichever is later. Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC. Patients having any condition that the treating physician believes would not be appropriate for immunosuppressive therapy should not receive treatment (e.g., absolute neutrophil count <1.3×10³/μL, platelet count <100×10³/μL, and hemoglobin <12 g/dL [male] or <10 g/dL [female]).

An alternative immune suppression regimen should be used on any patient who has any history of a hypersensitivity reaction to tacrolimus, sirolimus, or prednisolone. Patients with a history of primary immunodeficiency, splenectomy, or any underlying condition that predisposes the subject to infection should not be treated with immunosuppressive therapy. Patients with herpes zoster, cytomegalovirus, or Epstein-Barr Virus (EBV) infection that has not completely resolved for at least 12 weeks prior to screening should not be treated with immunosuppressive therapy. Patients with (1) any infection requiring hospitilization or treatment with parental anti-infectives not resolved at least 8 weeks prior to the second visit or (2) any active infection requiring oral anti-infectives (including antivirals) within ten days prior to the second visit or with a history of active tuberculosis or (3) a positive Quantiferon_TB Gold test during screening, or (4) any live vaccine within 8 weeks prior to signing the informed consent form, or (5) major surgery within 8 weeks before signing the informed consent or (6) major surgery planned during the study period should not be treated with immunosuppressive therapy. Patients with an absolute neutrophil count of <1.3×10³/μL should not be treated with immunosuppressive therapy.

Patients with a history of lymphoma or another cancer, other than squamous cell or basal cell carcinoma of the skin, should not be treated unless in full remission for at least 3 months before treatment.

Patients with uncontrolled hypertension (systolic BP >180 mmHg, diastolic BP >100 mmHg) despite maximal medical treatment should not be treated.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome and a fractionated bilirubin that shows conjugated bilirubin <35% of total bilirubin.

Patients with a history of infectious disease or substance abuse may not be candidates for treatment. For example, a history of human immunodeficiency virus (HIV) or hepatitis B or hepatitis C virus infection, or positive screening tests for hepatitis B surface antigen or hepatitis B core antibody, or hepatitis C or HIV antibodies; a history of alcohol or substance abuse within 1 year before treatment.

In one embodiment, the patients are adult patients. In another embodiment, the patients are pediatric patients.

Treatments Administered—Pre-Treatment with Immunosuppressive Therapy.

Prior to gene therapy, the patient should be treated with an immunosuppressive therapy to prevent immune responses to the transgene and/or AAV capsid. Such immunosuppressive therapy includes corticosteroids (methylprednisolone 10 mg/kg intravenously [IV] once on Day 1 predose and oral prednisone starting at 0.5 mg/kg/day on Day 2 with gradual tapering and discontinuation by Week 12), tacrolimus (1 mg twice daily [BID] by mouth [PO] Day 2 to Week 24 with target blood level of 4-8 ng/mL and tapering over 8 weeks between Week 24 and 32, and sirolimus (a loading dose of 1 mg/m² every 4 hours×3 doses on Day −2 and then from Day −1: sirolimus 0.5 mg/m²/day divided in BID dosing with target blood level of 4-8 ng/ml until Week 48. Neurologic assessments and tacrolimus/sirolimus blood level monitoring will be conducted. The doses of sirolimus and tacrolimus will be adjusted to maintain blood levels in the target range. No immunosuppression therapy is planned after week 48. In most subjects, dose adjustments can be based on the equation: new dose=current dose×(target concentration/current concentration). Subjects should continue on the new maintenance dose for at least 7-14 days before further dosage adjustment with concentration monitoring.

Gene Therapy.

A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

The rAAV9.hIDUA is administered as a single flat dose by IC administration: either a dose of 2×10⁹ GC/g brain mass (2.6×10¹² GC), or a dose of 1×10¹⁰ GC/g brain mass (1.3×10¹³ GC). The dose can be in a volume of about 5 to 20 ml.

For administration of rAAV9.IDUA, the subject is put under general anesthesia.

### 6.8 Example 8: Clinical Protocol Treatment of MPS I

The following example sets out a protocol that may be used to treat human subjects with a rAAV9.hIDUA vector to treat MPS I.

Patient Population.

Patients to be treated may include males or females 6 years or older who have:
a diagnosis of MPS I confirmed by enzyme activity, as measured in plasma, fibroblasts, or leukocytes (this includes those who may have previously or currently received HSCT or laronidase treatment).
early-stage neurocognitive deficit due to MPS I, defined as either of the following, if not explainable by any other neurologic or psychiatric factors:
   A score of ≥1 standard deviation below mean on IQ testing or in 1 domain of neuropsychological function (verbal comprehension, attention, or perceptual reasoning).
   A decline of >1 standard deviation on sequential testing.

Patients should have sufficient auditory and visual capacity, with or without aids, to complete the required protocol testing and willing to be compliant with wearing the aid, if applicable, on testing days.

Females of childbearing potential should have a negative serum pregnancy test on the day of treatment. All sexually active subjects must be willing to use a medically accepted method of barrier contraception from the screening visit until 24 weeks after vector administration. Sexually active females must be willing to use an effective method of birth control from the screening visit until 12 weeks after the last dose of sirolimus, whichever is later. Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC. Patients having any condition that the treating physician believes would not be appropriate for immunosuppressive therapy should not receive treatment (e.g., absolute neutrophil count <1.3×10³/μL, platelet count <100×10³/μL, and hemoglobin <12 g/dL [male] or <10 g/dL [female]).

An alternative immune suppression regimen should be used on any patient who has any history of a hypersensitivity reaction to tacrolimus, sirolimus, or prednisolone. Patients with a history of primary immunodeficiency, splenectomy, or any underlying condition that predisposes the subject to infection should not be treated with immunosuppressive therapy. Patients with herpes zoster, cytomegalovirus, or Epstein-Barr Virus (EBV) infection that has not completely resolved for at least 12 weeks prior to screening should not be treated with immunosuppressive therapy. Patients with (1) any infection requiring hospitilization or treatment with parental anti-infectives not resolved at least 8 weeks prior to the second visit or (2) any active infection requiring oral anti-infectives (including antivirals) within ten days prior to the second visit or with a history of active tuberculosis or (3) a positive Quantiferon_TB Gold test during screening, or (4) any live vaccine within 8 weeks prior to signing the informed consent form, or (5) major surgery within 8 weeks before signing the informed consent or (6) major surgery planned during the study period should not be treated with immunosuppressive therapy. Patients with an absolute neutrophil count of <1.3×10³/μL should not be treated with immunosuppressive therapy.

Patients with a history of lymphoma or another cancer, other than squamous cell or basal cell carcinoma of the skin, should not be treated unless in full remission for at least 3 months before treatment.

Patients with uncontrolled hypertension (systolic BP >180 mmHg, diastolic BP >100 mmHg) despite maximal medical treatment should not be treated.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome and a fractionated bilirubin that shows conjugated bilirubin <35% of total bilirubin.

Patients with a history of infectious disease or substance abuse may not be candidates for treatment. For example, a history of human immunodeficiency virus (HIV) or hepatitis B or hepatitis C virus infection, or positive screening tests for hepatitis B surface antigen or hepatitis B core antibody, or hepatitis C or HIV antibodies; a history of alcohol or substance abuse within 1 year before treatment.

Treatments Administered—Pre-Treatment with Immunosuppressive Therapy.

Prior to gene therapy, the patient should be treated with an immunosuppressive therapy to prevent immune responses to the transgene and/or AAV capsid. Such immunosuppressive therapy includes corticosteroids (methylprednisolone 10 mg/kg intravenously [IV] once on Day 1 predose and oral prednisone starting at 0.5 mg/kg/day on Day 2 with gradual tapering and discontinuation by Week 12), tacrolimus (1 mg twice daily [BID] by mouth [PO] Day 2 to Week 24 with target blood level of 4-8 ng/mL and tapering over 8 weeks between Week 24 and 32, and sirolimus (a loading dose of 1 mg/m² every 4 hours×3 doses on Day −2 and then from Day −1: sirolimus 0.5 mg/m²/day divided in BID dosing with target blood level of 4-8 ng/ml until Week 48. Neurologic assessments and tacrolimus/sirolimus blood level monitoring will be conducted. The doses of sirolimus and tacrolimus will be adjusted to maintain blood levels in the target range. No immunosuppression therapy is planned after week 48. In most subjects, dose adjustments can be based on the equation: new dose=current dose x (target concentration/current concentration). Subjects should continue on the new maintenance dose for at least 7-14 days before further dosage adjustment with concentration monitoring.

Gene Therapy.

A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

The rAAV9.hIDUA is administered as a single flat dose by IC administration: either a dose of 2×10⁹ GC/g brain mass (2.6×10¹² GC), or a dose of 1×10¹⁰ GC/g brain mass (1.3×10¹³ GC). The dose can be in a volume of about 5 to 20 ml.

For administration of rAAV9.IDUA, the subject is put under general anesthesia.

### 6.9 Example 9: Clinical Protocol Treatment of MPS I

The following example sets out a protocol that may be used to treat human subjects with a rAAV9.hIDUA vector to treat MPS I.

Patient Population.

Patients to be treated may include males or females 6 years or older and males or females younger than 3 years of age who have:
a diagnosis of MPS I confirmed by enzyme activity, as measured in plasma, fibroblasts, or leukocytes (this includes those who may have previously or currently received HSCT or laronidase treatment).
early-stage neurocognitive deficit due to MPS I, defined as either of the following, if not explainable by any other neurologic or psychiatric factors:
   A score of ≥1 standard deviation below mean on IQ testing or in 1 domain of neuropsychological function (verbal comprehension, attention, or perceptual reasoning).
   A decline of >1 standard deviation on sequential testing.
patients younger than 3 years of age have the severe form of MPS I (Hurler syndrome) confirmed by a mutation(s) known to lead to Hurler syndrome with neurocognitive decline.

Patients should have sufficient auditory and visual capacity, with or without aids, to complete the required protocol testing and willing to be compliant with wearing the aid, if applicable, on testing days.

Females of childbearing potential should have a negative serum pregnancy test on the day of treatment. All sexually active subjects must be willing to use a medically accepted method of barrier contraception from the screening visit until 24 weeks after vector administration. Sexually active females must be willing to use an effective method of birth control from the screening visit until 12 weeks after the last dose of sirolimus, whichever is later. Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC. Patients having any condition that the treating physician believes would not be appropriate for immunosuppressive therapy should not receive treatment (e.g., absolute neutrophil count <1.3×10³/μL, platelet count <100×10³/μL, and hemoglobin <12 g/dL [male] or <10 g/dL [female]).

An alternative immune suppression regimen should be used on any patient, or the patient should be excluded, who has any history of a hypersensitivity reaction to tacrolimus, sirolimus, or prednisolone. Patients with a history of primary immunodeficiency, splenectomy, or any underlying condition that predisposes the subject to infection should not be treated with immunosuppressive therapy. Patients with herpes zoster, cytomegalovirus, or Epstein-Barr Virus (EBV) infection that has not completely resolved for at least 12 weeks prior to screening should not be treated with immunosuppressive therapy. Patients with (1) any infection requiring hospitilization or treatment with parental anti-infectives not resolved at least 8 weeks prior to the second visit or (2) any active infection requiring oral anti-infectives (including antivirals) within ten days prior to the second visit or with a history of active tuberculosis or (3) a positive Quantiferon_TB Gold test during screening, or (4) any live vaccine within 8 weeks prior to signing the informed consent form, or (5) major surgery within 8 weeks before signing the informed consent or (6) major surgery planned during the study period should not be treated with immunosuppressive therapy. Patients with an absolute neutrophil count of <1.3×10³/μL should not be treated with immunosuppressive therapy.

Patients with a history of lymphoma or another cancer, other than squamous cell or basal cell carcinoma of the skin, should not be treated unless in full remission for at least 3 months before treatment.

Patients with uncontrolled hypertension (systolic BP >180 mmHg, diastolic BP >100 mmHg) despite maximal medical treatment should not be treated.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome and a fractionated bilirubin that shows conjugated bilirubin <35% of total bilirubin.

Patients with a history of infectious disease or substance abuse may not be candidates for treatment. For example, a history of human immunodeficiency virus (HIV) or hepatitis B or hepatitis C virus infection, or positive screening tests for hepatitis B surface antigen or hepatitis B core antibody, or hepatitis C or HIV antibodies; a history of alcohol or substance abuse within 1 year before treatment.

Treatments Administered—Pre-Treatment with Immunosuppressive Therapy.

Prior to gene therapy, the patient should be treated with an immunosuppressive therapy to prevent immune responses to the transgene and/or AAV capsid. Such immunosuppressive therapy, for patients 6 years or older, includes corticosteroids (methylprednisolone 10 mg/kg intravenously [IV] once on Day 1 predose and oral prednisone starting at 0.5 mg/kg/day on Day 2 with gradual tapering and discontinuation by Week 12), tacrolimus (1 mg twice daily [BID] by mouth [PO] Day 2 to Week 24 with target blood level of 4-8 ng/mL and tapering over 8 weeks between Week 24 and 32, and sirolimus (a loading dose of 1 mg/m² every 4 hours×3 doses on Day −2 and then from Day −1: sirolimus 0.5 mg/m²/day divided in BID dosing with target blood level of 4-8 ng/ml until Week 48. Such immunosuppressive therapy, for patients younger than 3 years of age, includes corticosteroids (methylprednisolone 10 mg/kg intravenously [IV] once on Day 1 predose and oral prednisone starting at 0.5 mg/kg/day on Day 2 with gradual tapering and discontinuation by Week 12), tacrolimus (0.05 mg/kg twice daily [BID] by mouth [PO] Day 2 to Week 24 with target blood level of 2-4 ng/mL and tapering over 8 weeks between Week 24 and 32, and sirolimus (a loading dose of 1 mg/m² every 4 hours×3 doses on Day −2 and then from Day −1: sirolimus 0.5 mg/m²/day divided in BID dosing with target blood level of 1-3 ng/ml until Week 48. Neurologic assessments and tacrolimus/sirolimus blood level monitoring will be conducted. The doses of sirolimus and tacrolimus will be adjusted to maintain blood levels in the target range. No immunosuppression therapy is planned after week 48. In most subjects, dose adjustments can be based on the equation: new dose=current dose x (target concentration/current concentration). Subjects should continue on the new maintenance dose for at least 7-14 days before further dosage adjustment with concentration monitoring.

Gene Therapy.

A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

For patients 6 years or older, rAAV9.hIDUA is administered as a single flat dose by IC administration: either a dose of 2×10⁹ GC/g brain mass (2.6×10¹² GC), or a dose of 1×10¹⁰ GC/g brain mass (1.3×10¹³ GC). The dose can be in a volume of about 5 ml or less.

For patients younger than 3 years of age, rAAV9.hIDUA is administered as a single flat dose by IC administration: either a dose of 1×10¹⁰ GC/g brain mass (6.0×10¹² GC for patients 4 months or older but younger than 9 months; 1.0×10¹³ GC for patients 9 months or older but younger than 18 months; 1.1×10¹³ GC for patients 18 months or older but younger than 3 years), or a dose of 5×10¹⁰ GC/g brain mass (3.0×10¹³ GC for patients 4 months or older but younger than 9 months; 5.0×10¹³ GC for patients 9 months or older but younger than 18 months; 5.5×10¹³ GC for patients 18 months or older but younger than 3 years). The dose can be in a volume of about 5 ml or less.

For administration of rAAV9.IDUA, the subject is put under general anesthesia.

### 6.10 Example 10: Clinical Protocol Treatment of MPS I

The following example sets out a protocol that may be used to treat human subjects with a rAAV9.hIDUA vector to treat MPS I.

Patient Population.

Patients to be treated may include males or females younger than 3 years of age who have:
a diagnosis of severe MPS I-Hurler confirmed by presence of clinical signs and symptoms compatible with MPS I-H, and/or homozygosity or compound heterozygosity for mutations exclusively associated with the severe phenotype.
an intelligent quotient (IQ) score of ≥55

Patients should have sufficient auditory and visual capacity, with or without aids, to complete the required protocol testing and willing to be compliant with wearing the aid, if applicable, on testing days.

Patients who may be excluded from intracisternal (IC) treatment can include subjects who have a contraindication for IC injection or lumbar puncture. Contraindications for an IC injection can include any of the following:
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR) <30 mL/min/1.73 m².

Patients who have received IT treatment at any time and experienced a significant adverse reaction considered related to IT administration should not be treated IC. Patients having any condition that the treating physician believes would not be appropriate for immunosuppressive therapy should not receive treatment (e.g., absolute neutrophil count <1.3×10³/μL, platelet count <100×10³/μL), and hemoglobin will be assessed.

An alternative immune suppression regimen should be used on any patient, or the patient should be excluded, who has any history of a hypersensitivity reaction to tacrolimus, sirolimus, or prednisolone. Patients with a history of primary immunodeficiency, splenectomy, or any underlying condition that predisposes the subject to infection should not be treated with immunosuppressive therapy. Patients with herpes zoster, cytomegalovirus, or Epstein-Barr Virus (EBV) infection that has not completely resolved for at least 12 weeks prior to screening should not be treated with immunosuppressive therapy. Patients with (1) any infection requiring hospitilization or treatment with parental anti-infectives not resolved at least 8 weeks prior to the second visit or (2) any active infection requiring oral anti-infectives (including antivirals) within ten days prior to the second visit or with a history of active tuberculosis or (3) a positive Quantiferon_TB Gold test during screening, or (4) any live vaccine within 8 weeks prior to signing the informed consent form, or (5) major surgery within 8 weeks before signing the informed consent or (6) major surgery planned during the study period should not be treated with immunosuppressive therapy. Patients with an absolute neutrophil count of <1.3×10³/μL should not be treated with immunosuppressive therapy.

Patients with a history of lymphoma or another cancer, other than squamous cell or basal cell carcinoma of the skin, should not be treated unless in full remission for at least 3 months before treatment.

Patients with uncontrolled hypertension (systolic BP >180 mmHg, diastolic BP >100 mmHg) despite maximal medical treatment should not be treated.

Patients having alanine aminotransferase (ALT) or aspartate aminotransferase (AST) >3×upper limit of normal (ULN) or total bilirubin >1.5×ULN should not be treated, unless the subject has a previously known history of Gilbert's syndrome.

Patients with a history of infectious disease or substance abuse may not be candidates for treatment. For example, a history of human immunodeficiency virus (HIV) or hepatitis B or hepatitis C virus infection, or positive screening tests for hepatitis B surface antigen or hepatitis B core antibody, or hepatitis C or HIV antibodies; a history of alcohol or substance abuse within 1 year before treatment.

Treatments Administered—Pre-Treatment with Immunosuppressive Therapy.

Prior to gene therapy, the patient should be treated with an immunosuppressive therapy to prevent immune responses to the transgene and/or AAV capsid. Prednisone dosing will start at 0.5 mg/kg/day and will be gradually tapered off by the Week 12 visit. Tacrolimus dose adjustments will be made to maintain whole blood trough concentrations within 2 to 4 ng/mL for the first 24 Weeks. At week 24 the dose will be decreased by approximately 50%. At Week 28 the dose will be further decreased by approximately 50%. Tacrolimus will be discontinued at Week 32. Sirolimus dose adjustments will be made to maintain whole blood trough concentrations within 1 to 3 ng/mL. In most subjects, dose adjustments can be based on the equation: new dose=current dose x (target concentration/current concentration). Subjects should continue on the new maintenance dose for at least 7 to 14 days before further dosage adjustment with concentration monitoring. See below for more details.

Corticosteroids

In the morning of vector administration (Day 1 predose), patients will receive methylprednisolone 10 mg/kg IV (maximum of 500 mg) over at least 30 minutes. The methylprednisolone should be administered before the lumbar puncture and IC injection of IP. Premedication with acetaminophen and an antihistamine is optional at the discretion of the investigator.

On Day 2, oral prednisone will be started with the goal to discontinue prednisone by Week 12. The dose of prednisone will be as follows:
Day 2 to the end of Week 2: 0.5 mg/kg/day
Week 3 and 4: 0.35 mg/kg/day
Week 5-8: 0.2 mg/kg/day
Week 9-12: 0.1 mg/kg
Prednisone will be discontinued after Week 12. The exact dose of prednisone can be adjusted to the next higher clinically practical dose.

Sirolimus

2 days prior to vector administration (Day −2): a loading dose of sirolimus 1 mg/m2 every 4 hours×3 doses will be administered

From Day −1: sirolimus 0.5 mg/m²/day divided in twice a day dosing with target blood level of 1-3 ng/ml

Sirolimus will be discontinued after the Week 48 visit.

Tacrolimus

Tacrolimus will be started on Day 2 (the day following IP administration) at a dose of 0.05 mg/kg twice daily and adjusted to achieve a blood level 2-4 ng/mL for 24 Weeks.

Starting at Week 24 visit, tacrolimus will be tapered off over 8 weeks. At week 24 the dose will be decreased by approximately 50%. At Week 28 the dose will be further decreased by approximately 50%. Tacrolimus will be discontinued at Week 32.

Gene Therapy.

A non-replicating recombinant AAV of serotype 9 capsid containing an hIDUA expression cassette (rAAV9.hIDUA) is used for treatment. The AAV9 serotype allows for efficient expression of the hIDUA protein in the CNS following IC administration. The vector genome contains an hIDUA expression cassette flanked by AAV2-inverted terminal repeats (ITRs). Expression from the cassette is driven by a strong constitutive CAG promoter. The rAAV9.hIDUA vector is suspended in Elliotts B solution for intrathecal injection.

The rAAV9.hIDUA is administered as a single flat dose by IC administration: either a dose of 1×10¹⁰ GC/g brain mass (6.0×10¹² GC for patients 4 months or older but younger than 9 months; 1×10¹³ GC for patients 9 months or older but younger than 18 months; 1.1×10¹³ GC for patients 18 months or older but younger than 3 years), or a dose of 5×10¹⁰ GC/g brain mass (3×10¹³ GC for patients 4 months or older but younger than 9 months; 5×10¹³ GC for patients 9 months or older but younger than 18 months; 5.5×10¹³ GC for patients 18 months or older but younger than 3 years). The dose can be in a volume of about 5 to 20 ml.

For administration of rAAV9.IDUA, the subject is put under general anesthesia.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1. A method for treating a human subject diagnosed with mucopolysaccharidosis I (MPS I), comprising delivering to the cerebrospinal fluid of the brain of said human subject a therapeutically effective amount of recombinant human α-L-iduronidase (IDUA) produced by human neuronal cells.

2. A method for treating a human subject diagnosed with MPS I, comprising delivering to the cerebrospinal fluid of the brain of said human subject a therapeutically effective amount of recombinant human IDUA produced by human glial cells.

3. The method of claim 1 or 2, further comprising administering an immune suppression therapy to said subject before or concurrently with the human IDUA treatment and continuing immune suppression therapy thereafter.

4. A method of treating a human subject diagnosed with MPS I, comprising:
delivering to the cerebrospinal fluid of the brain of said human subject, a therapeutically effective amount of a α2,6-sialylated human IDUA; and
administering an immune suppression therapy to said subject before or concurrently with the human IDUA treatment and continuing immune suppression therapy thereafter.

5. A method of treating a human subject diagnosed with mucopolysaccharidosis I (MPS I), comprising:
delivering to the cerebrospinal fluid of the brain of said human subject, a therapeutically effective amount of a glycosylated human IDUA that does not contain detectable NeuGc; and
administering an immune suppression therapy to said subject before or concurrently with the human IDUA treatment and continuing immune suppression therapy thereafter.

6. A method of treating a human subject diagnosed with mucopolysaccharidosis I (MPS I), comprising:
delivering to the cerebrospinal fluid of the brain of said human subject, a therapeutically effective amount of a glycosylated human IDUA that does not contain detectable NeuGc and/or α-Gal antigen; and
administering an immune suppression therapy to said subject before or concurrently with the human IDUA treatment and continuing immune suppression therapy thereafter.

7. A method of treating a human subject diagnosed with mucopolysaccharidosis I (MPS I), comprising:
delivering to the cerebrospinal fluid of the brain of said human subject, a therapeutically effective amount of human IDUA that contains tyrosine-sulfation; and
administering an immune suppression therapy to said subject before or concurrently with the human IDUA treatment and continuing immune suppression therapy thereafter.

8. A method of treating a human subject diagnosed with mucopolysaccharidosis I (MPS I), comprising:
administering to the brain of said human subject an expression vector encoding human IDUA, wherein said IDUA is α2,6-sialylated upon expression from said expression vector in a human, immortalized neuronal cell; and
administering an immune suppression therapy to said subject before or concurrently with the administration of the expression vector and continuing immune suppression therapy thereafter.

9. A method of treating a human subject diagnosed with mucopolysaccharidosis I (MPS I), comprising:
administering to the brain of said human subject an expression vector encoding human IDUA, wherein said IDUA is glycosylated but does not contain detectable NeuGc upon expression from said expression vector in a human, immortalized neuronal cell; and
administering an immune suppression therapy to said subject before or concurrently with the administration of the expression vector and continuing immune suppression therapy thereafter.

10. A method of treating a human subject diagnosed with mucopolysaccharidosis I (MPS I), comprising:
administering to the brain of said human subject an expression vector encoding human IDUA, wherein said IDUA is glycosylated but does not contain detectable NeuGc and/or α-Gal antigen upon expression from said expression vector in a human, immortalized neuronal cell; and
administering an immune suppression therapy to said subject before or concurrently with the administration of the expression vector and continuing immune suppression therapy thereafter.

11. A method of treating a human subject diagnosed with mucopolysaccharidosis I (MPS I), comprising:
administering to the brain of said human subject an expression vector encoding human IDUA, wherein said IDUA is tyrosine-sulfated upon expression from said expression vector in a human, immortalized neuronal cell; and
administering an immune suppression therapy to said subject before or concurrently with the administration of the expression vector and continuing immune suppression therapy thereafter.

12. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases said IDUA containing a α2,6-sialylated glycan; and
administering an immune suppression therapy to said subject before or concurrently with the administration of the expression vector and continuing immune suppression therapy thereafter.

13. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc; and
administering an immune suppression therapy to said subject before or concurrently with the administration of the expression vector and continuing immune suppression therapy thereafter.

14. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases glycosylated human IDUA that does not contain detectable NeuGc and/or α-Gal antigen; and
administering an immune suppression therapy to said subject before or concurrently with the administration of the expression vector and continuing immune suppression therapy thereafter.

15. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases said IDUA containing a tyrosine-sulfation; and
administering an immune suppression therapy to said subject before or concurrently with the administration of the expression vector and continuing immune suppression therapy thereafter.

16. The method of any one of claims 3 to 15 wherein the immune suppression therapy comprises administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment and continuing thereafter.

17. The method of claim 16 in which the immune suppression therapy is withdrawn after 180 days.

18. The method of any one of claims 1 to 17 in which the human IDUA comprises the amino acid sequence of SEQ ID NO. 1.

19. The method of claim 18 wherein the immune suppression therapy comprises administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment.

20. The method of claim 19 in which the immune suppression therapy is withdrawn after 180 days.

21. The method of claim 12 in which production of said IDUA containing a α2,6-sialylated glycan is confirmed by transducing a human neuronal cell line with said recombinant nucleotide expression vector in cell culture.

22. The method of claim 13 in which production of said glycosylated IDUA that does not contain detectable NeuGc is confirmed by transducing a human neuronal cell line with said recombinant nucleotide expression vector in cell culture.

23. The method of claim 14 in which production of said glycosylated IDUA that does not contain detectable NeuGc and/or α-Gal antigen is confirmed by transducing a human neuronal cell line with said recombinant nucleotide expression vector in cell culture.

24. The method of claim 15 in which production of said IDUA containing a tyrosine-sulfation is confirmed by transducing a human neuronal cell line with said recombinant nucleotide expression vector in cell culture.

25. The method of any one of claims 21-24, in which production is confirmed in the presence and absence of mannose-6-phosphate.

26. The method of any one of claims 8-15 and 21-25, or of any one of claims 16-17 when dependent directly or indirectly on any one of claims 8-15, wherein the expression vector or recombinant nucleotide expression vector encodes a signal peptide.

27. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases said IDUA containing a α2,6-sialylated glycan; and
administering an immune suppression therapy to said subject before or concurrently with the administration of the expression vector and continuing immune suppression therapy thereafter;
wherein said recombinant vector, when used to transduce human neuronal cells in culture results in production of said IDUA containing said α2,6-sialylated glycan in said cell culture.

28. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc; and
administering an immune suppression therapy to said subject before or concurrently with the administration of the expression vector and continuing immune suppression therapy thereafter;
wherein said recombinant vector, when used to transduce human neuronal cells in culture results in production of said IDUA that is glycosylated but does not contain detectable NeuGc in said cell culture.

29. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases glycosylated IDUA that does not contain detectable NeuGc and/or α-Gal antigen; and
administering an immune suppression therapy to said subject before or concurrently with the administration of the expression vector and continuing immune suppression therapy thereafter;
wherein said recombinant vector, when used to transduce human neuronal cells in culture results in production of said IDUA that is glycosylated but does not contain detectable NeuGc and/or α-Gal antigen in said cell culture.

30. A method of treating a human subject diagnosed with mucopolysaccharidosis I (MPS I), comprising:
administering to the cerebrospinal fluid of the brain of said human subject, a therapeutically effective amount of a recombinant nucleotide expression vector encoding human IDUA, so that a depot is formed that releases said IDUA that contains a tyrosine-sulfation; and
administering an immune suppression therapy to said subject before or concurrently with the administration of the expression vector and continuing immune suppression therapy thereafter;
wherein said recombinant vector, when used to transduce human neuronal cells in culture results in production of said IDUA that is tyrosine-sulfated in said cell culture.

31. The method of any of claims 27 to 30 wherein said immune suppression therapy comprises administering a combination of (a) tacrolimus and mycophenolic acid, (b) rapamycin and mycophenolic acid, or (c) tacrolimus, rapamycin, and a corticosteroid such as prednisolone and/or methylprednisolone to said subject before or concurrently with the human IDUA treatment and continuing thereafter.

32. The method of claim 31 in which the immune suppression therapy is withdrawn after 180 days.

33. The method of any one of claims 1-32, wherein the human subject is younger than 3 years of age.

34. The method of any one of claims 8-15 and 21-33, or of any one of claims 16-20 when dependent directly or indirectly on any one of claims 8-15, wherein the human subject is younger than 3 years of age and the expression vector or the recombinant nucleotide expression vector is administered at a dose of 1×10¹⁰ GC/g brain mass or 5×10¹⁰ GC/g brain mass.

35. The method of any one of claims 8-15 and 21-33, or of any one of claims 16-20 when dependent directly or indirectly on any one of claims 8-15, wherein the human subject is younger than 3 years of age and the expression vector or the recombinant nucleotide expression vector is administered at a dose ranging from 1×10¹⁰ GC/g brain mass to 5×10¹⁰ GC/g brain mass.
